(19) United States
(12) Patent Application Publication
(10) Pub. No.: US 2020/0058404 A1
(43) Pub. Date: Feb. 20, 2020

(54) INTERACTIVE COMPUTING SYSTEM TO GENERATE CUSTOMIZED PREVENTIVE HEALTH INFORMATION BASED ON AN INDIVIDUAL'S BIOMARKERS

(71) Applicant: Rejuvenan Global Health, Inc., New York, NY (US)

(72) Inventors: Fereydoun Fred Nazem, New York, NY (US); Thomas B. Okarma, Palo Alto, CA (US); Jeffrey T. Devine, New York, NY (US)

(21) Appl. No.: 16/335,184

(22) PCT Filed: Sep. 20, 2017
(86) PCT No.: PCT/US17/52502

§ 371 Date: Mar. 20, 2019

Related U.S. Application Data
(60) Provisional application No. 62/397,554, filed on Sep. 21, 2016.

(51) Int. Cl. G16H 50/30; G16H 50/20; G01N 33/68; G16H 10/60

(52) U.S. Cl. CPC G16H 50/30; G16H 50/20; G01N 33/6893; C12Q 2600/158; G01N 2800/60; G01N 2800/50; G16H 10/60

(57) ABSTRACT

The subject matter described herein generally relates to a computing system that can receive values of a plurality of biomarkers from a user, generate a score for each biomarker, compute a severity associated with each biomarker, generate an overall score for the user based on at least one of the score for each biomarker and the severity associated with each biomarker, generate treatment recommendations based on the score for each biomarker and the severity associated with each biomarker, and send those treatment recommendations to the user. The treatment recommendations can: 1) prevent or reduce disease progression within the user and the development of disease complications within the user, 2) reverse the disease or its complications within the user, and/or 3) reduce the need for medications the user is already taking for his/her condition. Related methods, techniques, systems, apparatuses, and articles are also described.

### CROSS-REFERENCE TO RELATED APPLICATION

The present application is a U.S. National Stage of International Patent Application No. PCT/US2017/052502, entitled “Interactive Computing System To Generate Customized Preventive Health Information Based On an Individual's Biomarkers” and filed Sep. 20, 2017. International Patent Application No. PCT/US2017/052502 claims the benefit of and priority to U.S. Provisional Patent Application Ser. No. 62/397,554, entitled “Interactive Computing System To Generate Customized Preventive Health Information Based On An Individual's Biomarkers” and filed Sep. 21, 2016, the entire contents of which are herein incorporated by reference.

### TECHNICAL FIELD

The subject matter described herein generally relates to a computing system that can receive values of a plurality of biomarkers from a user, generate a score for each biomarker, compute a severity associated with each biomarker, generate an overall score for the user based on at least one of the score for each biomarker and the severity associated with each biomarker, generate treatment recommendations based on the score for each biomarker and the severity associated with each biomarker, and send those treatment recommendations to the user. The treatment recommendations can be used to: 1) prevent or reduce disease progression within the user and the development of disease complications within the user, 2) reverse the disease or its complications within the user, and 3) reduce the need for medications the user is already taking for his/her condition.

### BACKGROUND

Many individuals in the country are at a risk of developing one or more diseases, such as obesity, hypertension, diabetes, cardiovascular diseases, poor nutrition, and so on. These individuals are also often prone to other complications, such as atherosclerosis, kidney disease, retinopathy, peripheral neuropathy, heart failure, chronic obstructive pulmonary disease, liver disease, and the like. A substantial set of individuals within this population fails to receive preventive care and/or care that addressing an existing condition, and ends up in the often uncomfortable or unsuccessful path of curing these diseases. Some conventional technologies provide generic educational guidance to the public, but do not provide customized guidance to individuals. Moreover, the traditional implementations are not interactive with users, but if they are then those interactions are not user-friendly, provide insufficient information without scientific justification, and are not prompt. Therefore, there exists a need to have a system and/or platform that can provide useful and customized information to a user in a timely manner and based on validated scientific justifications. The subject matter described herein addresses this need and provides additional benefits as well.

Throughout this specification, various patents, patent applications and other types of publications (for example, journal articles, electronic database entries, etc.) are referenced. The disclosure of all patents, patent applications, and other publications cited herein are hereby incorporated herein by reference in their entireties for all purposes.

### SUMMARY

In one aspect, a method for identifying a health score for a subject is described, as follows. One or more processors can receive one or more values corresponding to one or more biomarkers for a subject, which can be an individual. The one or more processors can execute a normalization routine to normalize each biomarker of the one or more biomarkers. The normalizing can quantify each biomarker on a preset scale corresponding to that biomarker to generate the normalized biomarker. The one or more processors can generate a score for each normalized biomarker of the one or more biomarkers. The one or more processors can obtain a predetermined weight for each normalized biomarker from a first database communicatively coupled to the one or more processors, and can then assign the predetermined weight to each normalized biomarker. The one or more processors can compute a health score for the subject based on the score for each normalized biomarker and the predetermined weight for each normalized biomarker.

In some variations, one or more of the following can be implemented either individually or in any feasible combination. The one or more processors can be located within a backend system. The one or more processors can receive the one or more values of the one or more biomarkers for the subject from at least one of a computing application executed on a computing device operably coupled with the backend system via a communication network and a second database operably coupled to the one or more processors. A part of the one or more processors that receives the one or more values of the one or more biomarkers from the computing application can be one of an application programming interface (API) module and a web module. A part of the one or more processors that performs the normalizing of each biomarker, the generating of the score for each biomarker, the obtaining of the predetermined weight for each biomarker, and the assigning of the predetermined weight to each biomarker can be a scoring module operably coupled to the API module and the web module.

At least one of the one or more biomarkers can be input on a computing application executed on a computing device operably coupled with the one or more processors via a communication network. At least one of the one or more biomarkers can be received from a second database storing a plurality of biomarkers previously input by the user on the computing application. The one or more biomarkers can be selected from a group consisting of: cholesterol level, waist to height ratio, blood pressure, serum A1C levels, alcohol consumption, glycemic food intake, nutrient dense food intake, physical activity level, smoking, frequency, and telomere length.

The one or more processors can assign a severity to each biomarker of the one or more biomarkers. The severity can be one of healthy, mild, moderate or severe. The one or more processors can generate a treatment recommendation based on the severity of each biomarker, on the score for each biomarker, and the score for the subject. The one or more processors can send the treatment recommendation to a computing device operably coupled with the one or more processors via a communication network. The treatment recommendation can include at least one of text and video. The treatment recommendation can be generated immediately after the receiving of the one or more values of the one or more biomarkers.

In another aspect, a method for reducing a likelihood of developing one or more physiological conditions is described, as follows. The one or more processors can receive a score for each biomarker of one or more biomarkers for an individual and a severity for each biomarker. The severity can be one of healthy, mild, moderate or severe. The one or more processors can remove at least one biomarker within the one or more biomarkers that corresponds to a predetermined set of severities. This at least one biomarker that is removed may neither have a severity assigned to it nor have a severity that does not fall within the predetermined set of severities. The one or more processors can analyze the one or more biomarkers after the removing of the at least one biomarker to determine a physiological condition associated with the score of each biomarker of the one or more biomarkers after the removing of the at least one biomarker. The one or more processors can generate a recommendation for improving the physiological condition. The one or more processors can transmit the recommendation to a computing application. The recommendation can be used to reduce the likelihood of the individual developing one or more physiological conditions.

In some variations of the aforementioned aspect, one or more of the following can be implemented either individually or in any feasible combination. The physiological condition can be cardiovascular disease, and the biomarker can be serum low density lipoprotein (LDL) level. Additionally or alternately, the physiological condition can be diabetes, and the biomarker is serum A1C level. Additionally or alternately, the physiological condition can be hypertension, and the biomarker can be systolic blood pressure. Additionally or alternately, the physiological condition can be obesity, and the biomarker can be waist to height ratio. Additionally or alternately, the physiological condition can be poor (for example, less than a threshold) activity, and the biomarker can be activity level. Additionally or alternately, the physiological condition can be excessive alcohol, and the biomarker can be alcohol consumption. Additionally or alternately, the physiological condition can be poor nutrition, and the biomarker can be high glycemic food intake and/or nutrient dense food intake. Additionally or alternately, the physiological condition can be smoking, and the biomarker can be smoking frequency.

Further, the physiological condition can be cardiovascular disease, and the recommendation for improving the physiological condition can be selected from the group consisting of diet modification, increased activity level, decreased alcohol consumption, or smoking cessation. Additionally or alternately, the physiological condition can be diabetes, and the recommendation for improving the physiological condition can be selected from the group consisting of diet modification, increased activity level, weight loss, or smoking cessation. Additionally or alternately, the physiological condition can be hypertension, and the recommendation for improving the physiological condition can be selected from the group consisting of increased activity level, meditation, decreased alcohol consumption, or smoking cessation. Additionally or alternately, the physiological condition can be obesity, and the recommendation for improving the physiological condition can be selected from the group consisting diet modification, increased activity level, or weight loss.

One or more of cardiovascular disease, diabetes, hypertension, or obesity can have biomarker scores with severe or moderate severities. The one or more processors can determining, by the one or more processors, that the individual has a co-morbid physiological condition associated with one or more of: a) cardiovascular disease, wherein the co-morbid physiological condition is one or more of poor activity, excessive alcohol, hypertension, obesity, or smoking when any of the co-morbid physiological conditions have biomarker scores with severe or moderate severities; b) diabetes, wherein the co-morbid physiological condition is one or more of poor activity, cardiovascular disease, poor nutrition due to high glycemic food intake, hypertension, obesity, or smoking when any of the co-morbid physiological conditions have biomarker scores with severe or moderate severities; c) hypertension, wherein the co-morbid physiological condition is one or more of poor activity, excessive alcohol, or smoking when any of the co-morbid physiological conditions have biomarker scores with severe or moderate severities; and d) obesity wherein the co-morbid physiological condition is smoking when the co-morbid physiological condition has a biomarker score with a severe or moderate severity.

In yet another aspect, a non-transitory computer program product is described that can storing instructions that, when executed by at least one programmable processor, cause the at least one programmable processor to perform at least the following operations: receiving one or more values corresponding to one or more biomarkers for a subject; executing a normalization routine to normalize each biomarker of the one or more biomarkers, the normalizing quantifying each biomarker on a corresponding preset scale to generate the normalized biomarker; generating a score for each normalized biomarker of the one or more biomarkers; assigning a predetermined weight for each normalized biomarker, the predetermined weight being obtained from a first database communicatively coupled to the at least one programmable processor; and computing a health score for the subject based on the generated score for each normalized biomarker and the predetermined weight of each normalized biomarker

In some variations of the aforementioned aspect, the one or more biomarkers can be selected from at least one of: cholesterol level, waist to height ratio, blood pressure, serum A1C levels, alcohol consumption, glycemic food intake, nutrient dense food intake, physical activity level, smoking, frequency and telomere length. The one or more biomarkers can form or constitute a biomarker panel.

In another aspect, a method of aiding in the reduction of a physiological condition of an individual is described, as follows. One or more processors can receive one or more values of one or more biomarkers specific for hypertension for an individual. The one or more processors can normalize each biomarker of the one or more biomarkers. The one or more processors can generate a score for each biomarker of the one or more biomarkers. The one or more processors can obtain a predetermined weight for each biomarker. The one or more processors can compute a health score for the individual based on the score for each biomarker and the predetermined weight for each biomarker.

In a further aspect, a system is described that can have a frontend unit and a content unit. The frontend unit can include one or more processors configured to: receive values of a plurality of biomarkers from an application executed by a computing device of a user, generate a score for each biomarker, compute a severity associated with each biomarker, and generate an overall score for the user based on at least one of the score for each biomarker and the severity associated with each biomarker. The content unit can be operably coupled to the frontend unit. The content unit can be configured to store a library of data. The content unit can include one or more processors configured to generate at least one treatment recommendation based on at least one of the score for each biomarker and the severity associated with each biomarker. The content unit can be configured to send the at least one treatment recommendation to the application.

In some variations of the aforementioned aspect, one or more of the following can be implemented either individually or in any feasible combination. The application can be configured to display the at least one treatment recommendation. The frontend unit can include a first cluster of instances. The content unit can include a second cluster of instances.

The system described above can further include an integrations unit. The integrations unit can include one or more processors operably coupled to the frontend unit. The computing device of the user can include a wearable device worn by the user. The integrations unit can be configured to receive at least one value of at least one biomarker of the plurality of biomarkers from the wearable device. The integrations unit can include a cluster of instances.

The system described above can also include an account and identity unit. The account and identity unit can include one or more processors operably coupled to the frontend unit. The account and identity unit can include authentication data associated with the user along with authentication data associated with a plurality of other users. The account and identity unit can include a cluster of instances.

The system described above can also include a secure health store unit. The secure health store unit can include one or more processors operably coupled to the frontend unit. The secure health store unit can store the values of the one or more biomarkers for the user. The secure health store unit can include a cluster of instances.

The system described above can also include a notifications unit. The notifications unit can include one or more processors operably coupled to the frontend unit. The notifications unit can be configured to generate a notification configured to be sent to the computing device of the user. The notifications unit can include a cluster of instances. The notification can be sent via at least one of an email, a text message, and a social network message. The notification can include an indication of the at least one treatment recommendation. The treatment recommendation can include an automated scheduling of a visit to a clinician.

Computer program products are also described that include non-transitory computer readable media storing instructions, which when executed by at least one data processors of one or more computing systems, causes at least one data processor to perform operations herein. Similarly, computer systems are also described that may include one or more data processors and a memory coupled to the one or more data processors. The memory may temporarily or permanently store instructions that cause at least one processor to perform one or more of the operations described herein. In addition, methods can be implemented by one or more data processors either within a single computing system or distributed among two or more computing systems.

The subject matter described herein provides many technical advantages. For example, the computing platform with an intuitive user-interface, personalized wellness contents and services can be easily accessed and implemented by the patient without the need for a healthcare provider. The present subject matter can be readily scaled to provide organizations and their employees tools to increase the overall health of the organizations as well as the individual employees. The implementations described herein can also provide analytical tools and data that can help the organizations and/or the individuals to make better financial decisions relating to their health. The current subject matter can allow an automation of a doctor's visit, thereby giving user more control of his/her life. The current subject matter can enable a user to get care twenty four hours a day and seven days every week, thereby putting the user in control of when that user should get care. Further, the described implementations can increase accuracy and reliability of a doctor's visit due to the automation enabled by those implementations. The implementations described herein can ensure accuracy and preciseness of medical processes and treatment recommendations.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a system diagram illustrating an exemplary computer-architecture of a system generating a health score and lifestyle recommendations for an individual based on biomarkers specific to that individual, according to some implementations of the current subject matter;

FIG. 2 is a flow diagram illustrating an exemplary calculation/computing/determining of health score for an individual based on biomarkers for that individual, according to some implementations of the current subject matter;

FIG. 3 is a flow diagram illustrating an exemplary collection and storage of current values for biomarkers of an individual, according to some implementations of the current subject matter;

FIG. 4 illustrates an exemplary screenshot of the application where the user can input data to receive a health score, according to some implementations of the current subject matter;

FIG. 5 is a flow diagram illustrating an exemplary process of a selection of biomarkers, values for which are interrogated from the user, according to some implementations of the current subject matter;

FIG. 6 is a flow diagram illustrating an exemplary sub-process of normalization of each biomarker within the process, according to some implementations of the current subject matter;

FIG. 7 is a flow diagram illustrating an exemplary sub-process of scoring each normalized biomarker within the process, according to some implementations of the current subject matter;

FIG. 8 is a flow diagram illustrating an exemplary process of assigning severity to each normalized biomarker, according to some implementations of the current subject matter;

FIG. 9 is a flow diagram illustrating an exemplary process of obtaining the predetermined weight for each biomarker, and using the weighted biomarkers for computing the overall score, according to some implementations of the current subject matter;

FIG. 10 is a flow diagram illustrating an exemplary process of calculating/computing/determining the health score, according to some implementations of the current subject matter;

FIG. 11 illustrates a screenshot of the application showing an exemplary health score of each biomarker for a user as well as the combined health score, according to some implementations of the current subject matter;

FIG. 12 is a flow diagram illustrating an exemplary process of collecting the current biomarkers for a user, according to some implementations of the current subject matter;

FIG. 13 is a flow diagram illustrating an exemplary process of collecting the current challenges faced by a user, according to some implementations of the current subject matter;

FIG. 14 is a flow diagram illustrating an exemplary process of loading of relevant programs based on health data, according to some implementations of the current subject matter;

FIG. 15 is a flow diagram illustrating an exemplary process of the removal of the content already displayed to the user, according to some implementations of the current subject matter;

FIG. 16 illustrates an exemplary list of content pieces arranged in an order in which they are displayed to a user, according to some implementations of the current subject matter;

FIG. 17 is a flow diagram illustrating an exemplary display of comorbidities, according to some implementations of the current subject matter;

FIG. 18 illustrates an exemplary graphical user interface displaying an email invitation for a user as sent by the system, according to some implementations of the current subject matter;

FIG. 19 illustrates an exemplary graphical user interface of the application displaying an overview of the application, according to some implementations of the current subject matter;

FIG. 20 illustrates an exemplary graphical user interface of the application displaying further overview of the application, according to some implementations of the current subject matter;

FIG. 21 illustrates an exemplary graphical user interface of the application displaying receipt of values of biomarkers to create a health profile of a user, according to some implementations of the current subject matter;

FIG. 22 illustrates another exemplary graphical user interface of the application displaying receipt of values of more biomarkers to create a health profile of a user, according to some implementations of the current subject matter;

FIG. 23 illustrates another exemplary graphical user interface of the application displaying receipt of values of more biomarkers to create a health profile of a user, according to some implementations of the current subject matter;

FIG. 24 illustrates an exemplary graphical user interface of the application displaying biomarker scores for the user and an overall health score for that user, according to some implementations of the current subject matter;

FIG. 25 illustrates an exemplary graphical user interface of the application displaying an interactive update for data associated with each biomarker, according to some implementations of the current subject matter;

FIG. 26 illustrates another exemplary graphical user interface of the application displaying another interactive update for data associated with each biomarker, according to some implementations of the current subject matter;

FIG. 27 illustrates an exemplary graphical user interface of the application displaying challenge programs recommended for the user based on the user's biomarker scores and the overall score, according to some implementations of the current subject matter;

FIG. 28 illustrates an exemplary graphical user interface of the application displaying details of a challenge program selected by the user, according to some implementations of the current subject matter;

FIG. 29 illustrates an exemplary graphical user interface of the application displaying details of a challenge program selected by the user, according to some implementations of the current subject matter;

FIG. 30 illustrates an exemplary graphical user interface of the application displaying further details of the weight challenge program, according to some implementations of the current subject matter;

FIG. 31 illustrates an exemplary graphical user interface of the application displaying a portion of a library of articles, recipes, videos, pictures, and any other data that are stored in the system and made available to a user, according to some implementations of the current subject matter;

FIG. 32 illustrates an exemplary graphical user interface of the application displaying another portion of the library of articles, recipes, videos, pictures, and any other data that are stored in the system and made available to a user, according to some implementations of the current subject matter; and

FIG. 33 illustrates another graphical user interface of the application displaying biomarker scores for the user and an overall health score for that user, according to some implementations of the current subject matter.

Like reference symbols in the various drawings indicate like elements.

### DETAILED DESCRIPTION

The subject matter described herein generally relates to a computing system that can receive values of multiple biomarkers from a computing device of a user, generate a score for each biomarker, compute a severity associated with the value for each biomarker, generate an overall score for the user based on at least one of the score for each biomarker and the severity associated with the value for each biomarker, generate treatment recommendations based on the score for each biomarker and the severity associated with the value for each biomarker, and send, subsequent (e.g., immediately) to the receipt of the values of biomarkers, those treatment recommendations to the computing device of the user. The treatment recommendations can: 1) prevent or reduce disease progression within the user and the development of disease complications within the user, 2) reverse the disease or its complications within the user, and 3) reduce the need for medications the user is already taking for his/her physiological condition. The physiological condition can be at least one of a cardiovascular disease, diabetes, hypertension, obesity, and other conditions. The treatment recommendation can include at least one of text and video. The treatment recommendations can be made continuously available on the computing device of the user for twenty four hours a day, seven days a week, and every day of the year. Related treatment methods, diagnostic methods and systems, computing methods, techniques, systems, apparatuses, articles, and biomarker panels are also described.

The implementations described herein are advantageous over traditional medical interventions. For example, the treatment recommendations provided to the user in the implementations described herein include behavioral and/or lifestyle changes that the user can adopt early in the course of the development of a physiological disease or condition, such as when signs and symptoms of the user's condition may be mild or even non-existent, or when the symptoms of a disease are present but not yet severe enough to warrant pharmaceutical intervention. These behavioral and/or lifestyle changes, if adopted by the user, can decrease or improve the severity of symptoms of the physiological condition and/or prevent the condition from progressing to a more severe state. Contrarily, the traditional medical interventions or traditional medicine does not allow for an early enough therapeutic intervention for a certain physiological diseases or conditions compared to that normally used in traditional medicine.

### Definitions

A “biomarker” used herein refers to any measurement related to the biological system of an individual being assessed and/or treated. It can include, but is not limited to, measurement of molecules (for example, proteins, serum cholesterol levels) in a sample from such individual, information provided by an individual (for example, age, height, waist size, blood pressure, etc.) and actions that the individual takes (for example, consumption of certain foods, physical activity, etc.).

As used herein, “reducing a likelihood of developing” a particular physiological condition or disease means to delay and/or postpone development of the physiological condition or disease. This delay can be of varying lengths of time, depending on the history of the disease and/or individual being treated. As is evident to one skilled in the art, a sufficient or significant delay can, in effect, encompass prevention, in that the individual does not develop the physiological condition or disease. A method that reduces a likelihood of developing one or more physiological conditions is a method that reduces the probability of disease development in a given time frame and/or reduces the extent of the physiological condition or disease or its complications in a given time frame, when compared to not using the method. Such comparisons are typically based on studies using a statistically significant number of subjects. “Developing” may also refer to disease progression that may be initially undetectable and includes occurrence, recurrence, and onset.

As used herein, the phrase “aiding in the reduction of a physiological condition” means any of decreasing or reducing one or more symptoms of a physiological condition (such as, a chronic disease), preventing an individual from developing a physiological condition (such as, an individual predisposed for developing a physiological condition, such as a chronic disease) and/or reducing the likelihood that an individual will develop a physiological condition (such as a chronic disease). In some embodiments, the systems, methods, non-transitory computer programmable products, and/or articles described herein aid in the reduction of one or more physiological conditions such as, but not limited to, chronic diseases including cardiovascular disease, diabetes, hypertension, and/or obesity.

As used herein, the term “individual” or “subject” or “user” refers to a vertebrate, such as a mammal or a human. Mammals include, but are not limited to, murines, simians, humans, farm animals, sport animals, companion animals, and pets.

As used herein, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

Reference to “about” a value or parameter herein includes (and describes) variations that are directed to that value or parameter per se. For example, description referring to “about X” includes description of “X+/−5% of X.”

As used herein, the singular terms “a,” “an,” and “the” may include the plural reference unless the context clearly indicates otherwise.

A composition or method described herein as “comprising” or “including” one or more named elements or steps is open-ended, meaning that the named elements or steps are essential, but other elements or steps may be added within the scope of the composition or method. To avoid prolixity, it is also understood that any composition or method described as “comprising” or “including” (or “comprises” or “includes”) one or more named elements or steps also describes the corresponding, more limited, composition or method “consisting essentially of” (or “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and close-ended composition or method “consisting of” (or “consists of”) the named elements or steps to the exclusion of any other unnamed element or step. In any composition or method disclosed herein, known or disclosed equivalents of any named essential element or step may be substituted for that element or step.

Unless defined otherwise herein, all technical and scientific terms used herein have the same meaning as commonly understood by one of ordinary skill in the art to which the current subject matter pertains.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Systems Involving Biomarkers

The systems and/or platforms described herein utilize certain biomarkers (including combination of biomarkers) for assessment and/or diagnosis of certain physiological conditions in individuals. Further actions can be taken to address the physiological conditions so that there is an improvement in that individual (e.g., reduction of a symptom) as detailed below and herein.

Biomarkers that can be used to assess an individual's health include, but are not limited to, cholesterol level (for example, low density lipoprotein (LDL) levels or high density lipoprotein (HDL) levels), waist to height ratio, blood pressure, serum A1C levels, alcohol consumption, glycemic food intake, nutrient dense food intake, physical activity level, smoking, and telomere length.

Elevated levels of cholesterol, in particular LDL and triglycerides in the blood, have been associated with the development of fatty plaques, which can lead to generalized vascular damage, atherosclerosis and eventually heart attack. The term “cholesterol” as used herein refers to the monohydric alcohol form, which is a white, powdery substance that is found in all animal cells and in animal-based foods (not in plants). The term “lipoproteins” as used herein are protein spheres that transport cholesterol, triglyceride, or other lipid molecules through the bloodstream. Lipoproteins are categorized into types according to size and density. They can be further defined by whether they carry cholesterol (high density lipoproteins (HDL) and low density lipoproteins (LDL)) or triglycerides (intermediate density lipoproteins (IDL), very low density lipoproteins (VLDL), and chylomicrons)). Atherosclerosis is a leading form of cardiovascular disease, which involves the slow build-up of fatty plaques on the arterial wall. This build-up can damage the vascular endothelium causing inflammation, a narrowing of the arteries and potential arterial blockages that can result in heart attacks. Cholesterol levels in many people can be controlled by diet, but for many patients diet changes alone are insufficient to reduce high cholesterol. Cholesterol lowering drugs such as Zocor® (simvastatin) and Lipitor® (atorvastatin) can be prescribed to help patients lower their cholesterol levels. Serum cholesterol levels (such as LDL levels) can be measured by any means known in the art. Cholesterol is typically measured as milligrams per deciliter (mg/dL) of blood in the United States and some other countries. In the United Kingdom, most European countries, and Canada, millimoles per liter of blood (mmol/L) is the most commonly used measure.

Blood pressure (BP) is the pressure exerted by circulating blood upon the walls of blood vessels. As used herein, “blood pressure” refers to the arterial pressure in the systemic circulation. Blood pressure is usually expressed in terms of the systolic (maximum) pressure over diastolic (minimum) pressure and is measured in millimeters of mercury (mm Hg). In some embodiments, a normal resting systolic (diastolic) blood pressure in an adult is approximately 120 mm Hg (80 mm Hg), abbreviated “120/80 mm Hg.” Blood pressure can be assessed by any means known in the art but is most commonly measured non-invasively via a sphygmomanometer.

A waist-to-height ratio (WHtR), also called waist-to-stature ratio (WSR), is defined as an individual's waist circumference divided by their height, both measured in the same units. The WHtR is a measure of the distribution of body fat. Higher values of WHtR are correlated with a higher risk of obesity-related cardiovascular diseases as well as with abdominal obesity.

Glycated hemoglobin (also known as hemoglobin A1C; sometimes also referred to as being Hb1c or HGBA1C) is a form of hemoglobin that is measured primarily to identify the three-month average plasma glucose concentration. The test is limited to a three-month average because the lifespan of a red blood cell is four months (120 days). Glycated hemoglobin is formed in a non-enzymatic glycation pathway by hemoglobin's exposure to plasma glucose. Normal levels of glucose produce a normal amount of glycated hemoglobin. As the average amount of plasma glucose increases, the fraction of glycated hemoglobin increases in a predictable way. As such, A1C is a biomarker for average blood glucose levels over the previous three months before the measurement. In individuals diagnosed with or predisposed to developing diabetes mellitus, higher A1C indicates poorer control of blood glucose levels and is also associated with conditions such as cardiovascular disease, nephropathy, neuropathy, and retinopathy. Any method known in the art can be used to determine serum A1C levels such as, but not limited to, high-performance liquid chromatography (HPLC), immunoassays, enzymatic assays, capillary electrophoresis, and/or boronate affinity chromatography.

Alcohol consumption refers to the daily intake or consumption of alcoholic beverages and is typically measured via self-reporting. However, alcohol consumption can also be measured using blood tests and/or devices capable of detecting alcohol consumption via an individual's breath (i.e. a breathalyzer).

“Glycemic food intake,” as used herein, refers consumption of food or food ingredient and the subsequent effect of that food or food ingredient on blood sugar (glucose), A1C, and/or insulin levels. Whether a food is considered “high” or “low” for purposes of glycemic food intake can be determined based on a “glycemic index” (GI) established for the food. A food's glycemic index is determined relative to the effect of consuming pure glucose. Foods with carbohydrates that break down quickly during digestion and which release glucose rapidly into the bloodstream tend to have a high GI; foods with carbohydrates that break down more slowly, releasing glucose more gradually into the bloodstream, tend to have a low GI. Glycemic food intake is typically self-reported.

“Nutrient dense food intake,” as used herein, refers to consumption of food having a relatively high proportion of nutrients relative to other foods. Nutrient-dense foods such as fruits and vegetables are the opposite of energy-dense food (also called “empty calorie” food), such as alcohol and foods high in added sugar or processed cereals. Further, nutrient-dense foods are excellent sources of vitamins or minerals such as the B-vitamins, vitamins A, C, D and E, protein, calcium, iron, potassium, zinc, fiber and monounsaturated fatty acids. Nutritional rating systems are methods of ranking or rating food products or food categories to communicate the nutritional density of food in a simplified manner to a target audience. Rating systems have been developed by governments, nonprofit organizations, or private institutions and companies. Such rating systems can be used in accordance with the methods disclosed herein to determine types of nutrient dense foods for determination of nutrient dense food intake. These rating systems can include, without limitation, Guiding Stars (see Canadian Patent No. 2,652,379, incorporated herein by reference in its entirety), Nutripoints, Nutrition iQ, NuVal® Nutrition Scoring System, Aggregate Nutrient Density Index (ANDI), or Naturally Nutrient Rich (NNR; Drewnowski, Adam. “Concept of a nutritious food: toward a nutrient density score” Am J Chu Nutr October 2005 vol. 82 no. 4; 721-7).

Physical activity level (PAL) is a way to express a person's daily physical activity as a number, and is used to estimate a person's total energy expenditure. In combination with the basal metabolic rate, it can be used to compute the amount of food energy a person needs to consume in order to maintain a particular lifestyle. In some embodiments, physical activity level is defined for a non-pregnant, non-lactating adult as the total energy expenditure (TEE) in a 24-hour period, divided by his or her basal metabolic rate (BMR). Physical activity level is typically self-reported. However, in some embodiments, PAL can be determined at least in part from smart and/or wearable devices (for example, APPLE watch, FITBIT, etc.), and/or other personal health monitoring devices. In some alternate implementations, PAL can be determined at least in part from at least one of: (a) one or more backend computing systems communicatively coupled to the smart and/or wearable devices and/or personal health monitoring devices, and (b) a computing device—such as a laptop computer, a desktop computer, a tablet computer, a cellular smart phone, a phablet, a computing kiosk, and/or any other computing device, which in some exemplary non-limiting embodiments, can be communicatively coupled to the smart and/or wearable devices and/or personal health monitoring devices.

Smoking is one of the most common forms of recreational drug use. Tobacco smoking is the most popular form, being practiced by over one billion people globally, of whom the majority are in the developing world. Smoking behavior and frequency is typically a self-reported biomarker in accordance with the methods disclosed herein.

Telomeres are specialized protein-bound DNA structures at the ends of eukaryotic chromosomes that appear to function in chromosome stabilization, positioning, and replication. In all vertebrates, telomeres consist of hundreds to thousands of tandem repeats of a 5′-TTAGGG-3′ sequence and associated proteins. In all normal somatic cells examined to date, chromosomes lose about 50-200 nucleotides of telomeric sequence per cell division, consistent with the inability of DNA polymerase to replicate linear DNA to the ends. This shortening of telomeres has been proposed to be the mitotic clock by which cells count their divisions, and a sufficiently short telomere(s) may be the signal for replicative senescence in normal cells. Telomere length can be determined using any means known in the art including, without limitation, analysis of chromosome terminal restriction fragments (TRF). In some implementations, the present subject matter can provide customized digital interventions based on one or more biomarkers. These biomarkers can include one or more of the following examples including one or more routine health behaviors, physical attributes, and blood tests. Based on the biomarker(s), the present subject matter can assess the user's health and identify one or more physiologic conditions or diseases and/or their severity and/or the likelihood of developing a complication of the physiologic conditions or diseases. The biomarker data is used to generate an individually specific set of behavioral treatments (for example, in the form of videos and/or SMS texts) known in the medical literature to: 1) prevent or reduce disease progression and the development of disease complications, 2) reverse the disease or its complications and/or 3) reduce the need for medications the user is already taking for his/her condition.

In some implementations, data representing the one or more biomarkers can be entered manually. Some of the data can be synched from smart and/or wearable devices (for example, APPLE watch, FITBIT, etc.), and/or other personal health monitoring devices. Further, some of the data can be alternately or additionally be synched from at least one of: (a) one or more backend computing systems communicatively coupled to the smart and/or wearable devices and/or personal health monitoring devices, and (b) a computing device—such as a laptop computer, a desktop computer, a tablet computer, a cellular smart phone, a phablet, a computing kiosk, and/or any other computing device, which, in some exemplary non-limiting embodiments, can be communicatively coupled to the smart and/or wearable devices and/or personal health monitoring devices.

Diseases or Physiological Conditions Associated with Biomarkers

As shown in Table 1, each of the biomarkers assessed in accordance with the methods described herein maps to or is associated with one or more diseases or physiological conditions that adversely affect health.

TABLE 1

| Biomarker | Disease or Physiological Condition |
|---|---|
| Physical Activity Level | Poor activity; sedentary lifestyle |
| Cholesterol (for example, | Cardiovascular disease |
| LDL or HDL) | |
| A1C | Diabetes (such as type 2 diabetes); pre- |
| | diabetes; metabolic syndrome |
| Alcohol | Excessive alcohol consumption |
| Blood pressure (for example, | Hypertension |
| systolic and/or diastolic) | |
| High glycemic food intake | Poor nutrition |
| Low nutrient dense food intake | Poor nutrition |
| Waist to height ratio | Obesity |
| Smoking | Smoking-related illness |
| Telomere length | Cellular aging/senescence |

A lack of physical activity is one of the leading causes of preventable death worldwide. As used herein, individuals who have no or irregular physical activity are said to be engaging in a “sedentary lifestyle.” Lack of exercise causes muscle atrophy, i.e. shrinking and weakening of the muscles and accordingly increases susceptibility to physical injury. Additionally, regular physical activity is correlated with immune system function and decreased development of cardiovascular and endocrine-related disorders.

“Cardiovascular disease,” as used herein, refers to a class of diseases that involve the heart or blood vessels and can include coronary artery diseases (CAD) such as angina and myocardial infarction (commonly known as a heart attack). Complications associated with cardiovascular diseases can include, without limitation, stroke, hypertensive heart disease, rheumatic heart disease, cardiomyopathy, heart arrhythmia, congenital heart disease, valvular heart disease, carditis, aortic aneurysms, peripheral artery disease, and venous thrombosis. Cardiovascular diseases are the leading cause of death globally (Mendis et al., World Health Organization (2011). Global Atlas on Cardiovascular Disease Prevention and Control. World Health Organization in collaboration with the World Heart Federation and the World Stroke Organization. pp. 3-18).

Diabetes mellitus (DM), commonly referred to as diabetes, is a group of metabolic diseases in which there are high blood sugar levels over a prolonged period. Symptoms of high blood sugar include frequent urination, increased thirst, and increased hunger. If left untreated, diabetes can cause complications which can include, without limitation, diabetic ketoacidosis, nonketotic hyperosmolar coma, or death. Serious long-term complications include heart disease, stroke, chronic kidney failure, foot ulcers, and damage to the eyes (for example, retinopathy). In some embodiments, they type of diabetes is type 2 diabetes. Type 2 diabetes begins with insulin resistance, a condition in which cells fail to respond to insulin properly. As the disease progresses a lack of insulin production by the pancreas may also develop. The primary cause of type 2 diabetes is excessive body weight and not enough physical activity.

The long-term effects of alcohol consumption range from cardioprotective health benefits for low to moderate alcohol consumption in industrialized societies to higher rates of cardiovascular disease to severe detrimental effects in cases of chronic alcohol abuse. Complications associated with large levels of alcohol intake include an increased risk of alcoholism, malnutrition, chronic pancreatitis, alcoholic liver disease and cancer. In addition, damage to the central nervous system and peripheral nervous system can occur from chronic alcohol abuse. The long-term use of alcohol is capable of damaging nearly every organ and system in the body.

Consistently high blood pressure is known as hypertension. High blood pressure usually does not cause symptoms. However, long term high blood pressure, is a major risk factor for complications such as, without limitation, coronary artery disease, stroke, heart failure, peripheral vascular disease, vision loss, and chronic kidney disease.

As used herein, “poor nutrition” refers to consistent consumption of both foods with a high glycemic index as well as low nutrient density. Chronic consumption of a diet with a high glycemic index is independently associated with complications such as increased risk of developing type 2 diabetes, cardiovascular disease, and certain cancers. Further, nutritional deficiencies (such as, but not limited to, vitamin and mineral deficiencies), are associated with a number of diseases and conditions as well as a predisposition for developing cardiovascular diseases and/or diabetes.

“Obesity,” as used herein refers to a medical condition in which excess body fat has accumulated to the extent that it has a negative effect on health. Complications associated with excessive body weight include cardiovascular diseases, diabetes mellitus type 2, obstructive sleep apnea, certain types of cancer, osteoarthritis, and asthma. As a result, obesity has been found to reduce life expectancy. Obesity is most commonly caused by a combination of excessive food intake, lack of physical activity, and genetic susceptibility.

Smoking generally has negative health effects, because smoke inhalation inherently poses challenges to various physiologic processes such as respiration. Diseases and complications related to tobacco smoking have been shown to kill approximately half of long term smokers when compared to average mortality rates faced by non-smokers. A 2007 report states that, each year, about 4.9 million people worldwide die as a result of smoking (West, Robert; Shiffman, Saul (2007). Fast Facts: Smoking Cessation. Health Press Ltd. p. 28).

Cellular aging or senescence is the phenomenon by which normal diploid cells cease to divide. In culture, fibroblasts can reach a maximum of 50 cell divisions before becoming senescent. This phenomenon is known as “replicative senescence.” Replicative senescence is the result of telomere shortening that ultimately triggers a DNA damage response.

### Biomarker Scores

In some implementations, and as discussed further below, a score of 0-100 (for example) is assigned for each biomarker. In some embodiments, the biomarker score is indicative of the severity of the specific condition known in the art to be associated with one or more diseases or physiological conditions. A lower score correlates with a more severe condition as well as a higher risk for the development of complications associated with that condition (for example, a lower biomarker score for the serum A1C biomarker correlates with more severe diabetes as well as a higher risk of developing diabetes-related complications such as, but not limited to, retinopathy and/or peripheral neuropathy). In some embodiments, a score of 76-100 indicates that the individual is healthy for a given biomarker. A score of 51-75 indicates that an individual has a mild risk of developing one or more diseases or conditions associated with that particular biomarker. A scope of 26-50 indicates that an individual has a moderate risk of developing one or more diseases or conditions associated with a particular biomarker. Further, a score of 0-25 indicates that an individual has a severe risk of developing one or more diseases or conditions associated with a particular biomarker. In other embodiments, the score can be configured to represent the likelihood (for example, based on current medical literature or research) of a particular biomarker contributing to the user's risk of developing one or more diseases or conditions or whether an individual currently has one or more diseases or conditions.

In some implementations, the present subject matter can be further configured to determine an overall health score (“Overall Health Score” or OHS), which can represent an assessment of the overall health and wellness like a personal credit score. The computing of the OHS is described in detail below. In some implementations, the higher the OHS score the healthier the individual and the less likely the individual will have or be at risk for the development of chronic diseases or conditions and/or complications associated with those conditions.

The methods described herein can use inputted biomarker scores to identify one or more comorbid conditions, as is described in detail below. As used herein, the term “comorbid” means that at least two diseases or conditions coexist or are found in the same individual. For example, comorbid cardiovascular disease and hypertension means that cardiovascular disease and hypertension coexist or are found in the same subject, i.e., that a single subject experiences, tends to experience, or has a history of experiencing, both cardiovascular disease and hypertension.

In some embodiments, comorbid conditions are identified by first determining whether the biomarker scores for one or more of cardiovascular disease, diabetes, hypertension, and/or obesity are indicative of moderate or severe risk (i.e. whether the biomarker scores for one or more of these physiological conditions are between 0-50). If one or more of these conditions are scored as indicative of moderate to severe risk, comorbid conditions are identified based on risk biomarker scores of moderate or severe of the comorbid conditions shown in Table 2.

TABLE 2

| Main condition | Comorbid condition(s) |
|---|---|
| Cardiovascular disease | poor activity |
| | excess alcohol |
| | hypertension |
| | obesity |
| | smoking |
| Diabetes | poor activity |
| | cardiovascular disease |
| | poor nutrition |
| | (via high glycemic foods biomarker) |
| | hypertension |
| | obesity |
| | smoking |
| Hypertension | poor activity |
| | excess alcohol |
| | smoking |
| Obesity | hypertension |

If any of the comorbid conditions listed in Table 2 are assigned a biomarker score indicative of moderate or severe risk, then the main condition is determined to be comorbid with the comorbid condition. For example, if an individual has a biomarker score indicative of moderate hypertension and a biomarker score of severe excess alcohol consumption, then both hypertension and excess alcohol consumption would be considered to be comorbid conditions. In contrast, if an individual has a biomarker score indicative of moderate hypertension and a biomarker score of mild excess alcohol consumption, then there would be no comorbid conditions. As a further non-limiting example, if an individual has a biomarker score indicative of moderate hypertension, a biomarker score of severe excess alcohol consumption, and a biomarker score of severe smoking, then the individual has comorbidity for hypertension and excess alcohol consumption as well as comorbidity for hypertension and smoking.

### Computing Platform and its Uses

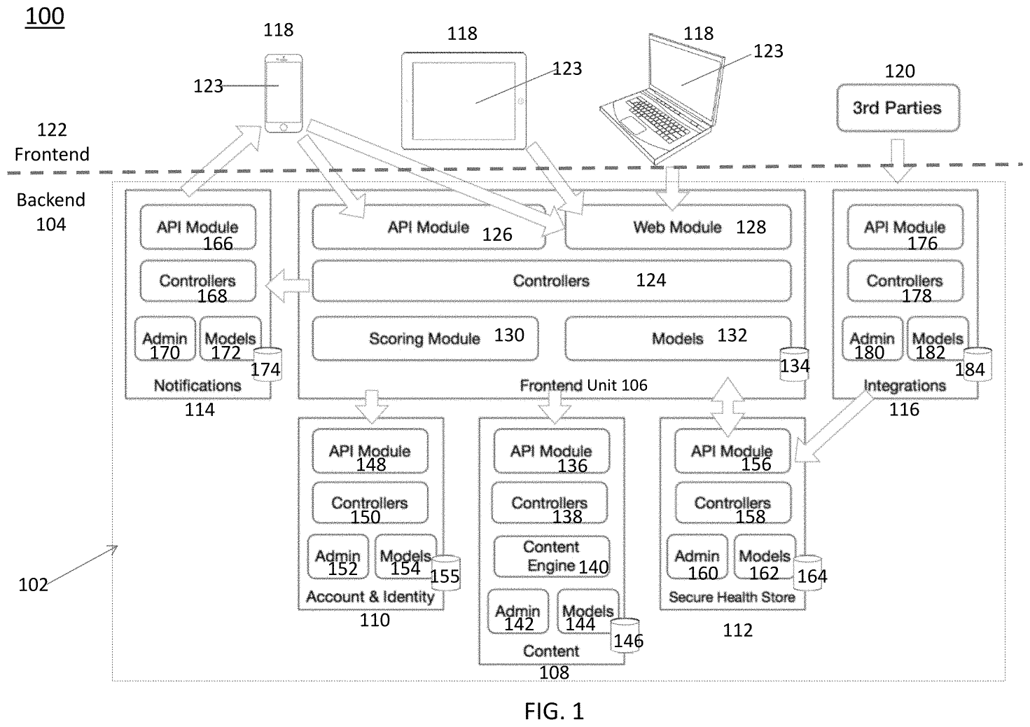

The current subject matter also provides for systems and platforms for its practice, such as a computing platform. As a non-limiting illustration of the current subject matter, FIG. 1 is a system diagram illustrating a computer-architecture 100 of a system 102 generating a health score and lifestyle recommendations for an individual based on biomarkers specific to that individual. The system 102 can be located at the backend 104, and can include a frontend unit 106, a content unit 108, an account and identity unit 110, a secure health store unit 112, a notifications unit 114, and an integrations unit 116. The system 102 can communicate with computing devices 118 and third party systems 120 located at the frontend 122 via a communication network. The system 102 can control an application 123 that can be displayed on the computing devices 118 and the third party systems 120.

A biomarker can be any measurement related to the biological system of an individual being assessed and/or treated. It can include, for example, measurement of molecules, such as proteins or cholesterol levels (such as, LDL cholesterol levels), in a sample from the user. Further, biomarkers can also or alternately include actions that the individual takes, such as consumption of certain foods, physical activity, and the like. These biomarkers can be measured by any means known to one of skill in the art. In some implementations, the system 102 and application 123 can provide customized digital interventions based on certain biomarkers, such as routine health behaviors, physical attributes, blood tests, and the like. In some implementations, data representing the one or more biomarkers can be entered manually by either the user of the computing device 114 or an authorized administrator of the system 102. Some of the data can be synched from smart and/or wearable devices (for example, APPLE watch, FITBIT, etc.), and/or other personal health monitoring devices. Further, some of the data can be alternately or additionally be synched from at least one of: (a) one or more backend computing systems communicatively coupled to the smart and/or wearable devices and/or personal health monitoring devices, and (b) a computing device—such as a laptop computer, a desktop computer, a tablet computer, a cellular smart phone, a phablet, a computing kiosk, and/or any other computing device, which, in some exemplary non-limiting embodiments, can be communicatively coupled to the smart and/or wearable devices and/or personal health monitoring devices.

The frontend unit 106 can include one or more controllers 124, an application programming interface (API) module 126, a web module 128, a scoring module 130, one or more models 132 and a database 134. The frontend unit 106 can be one or more instances, each of which can also be referred to as a virtual server. The API module 126 can receive data from the application 123. At least some of this data may be input by the user on the application 123. The application 123 can also be made available over the web or internet, and in that implementation the web module 128 can receive data from the application 123 when accessed by the computing devices 118 over the internet. The one or more controllers 124 can process data, including requests, from the computing devices 118 and the third party systems 120. The database 134 can store data associated with each user separately. The scoring module 130 can receive the biomarker data associated with an individual either from the database 134, or directly from one of the API module 126 and the web module 128.

The scoring module 130 can then use the data to score all biomarkers, and then use the scores for the biomarkers to compute an overall health score. The score for each biomarker can represent the severity, based on the current medical literature or research, of a particular disease or physiological condition known to be associated with a particular biomarker (for example, the serum A1C biomarker is associated with diabetes). Each model 132 can be a collection of user-specific data that can identify the user uniquely, such as a username and/or password of that user. The model 132 can further store all biomarker data for that user. The model 132 can have a one to one mapping with tables that are associated with that user and are stored in the database 134. The one or more models 132 can facilitate the creation and use of business objects whose data requires persistent storage to a database 134. The one or more models 132 may only interact with the database 134.

The frontend unit 106 can proxy all API calls to a relevant unit, which is one of the content unit 108, the account and identity unit 110, the secure health store unit 112, the notifications unit 114, and the integrations unit 116. In one implementation, the frontend unit 106 can be a cluster of instances, such as EC2 instances. The EC2 instance can be a virtual server in AMAZON's Elastic Compute Cloud (EC2) for running applications on the AMAZON WEB SERVICES (AWS) infrastructure. Each instance of the frontend unit 106 can be a virtual server, which can be scaled and deployed independently of the other units. Although the frontend unit 106 has been described as a virtual server, in an alternate implementation the frontend unit 106 can be a physical server, which can be a co-located server, an on-premise server, a collection of servers, and/or any other type of servers and/or any combination thereof. The one or more physical servers can be communicatively coupled using at least one of the following a wireless network, a wired network, a metropolitan area network, a wide area network, a local area network, a virtual local area network, and/or any other type of network and/or any combination thereof. The scaling of an instance of the frontend unit 106 refers to launching one or more identical instances to allow more compute capacity by the frontend unit 106. For example, if there is a significant spike in traffic (for example, number of users accessing the respective applications 123), the frontend unit 106 can handle the load by scaling horizontally to include more instances to handle the traffic.

The content unit 108 can include an API module 136, one or more controllers 138, a content engine 140, an admin module 142, models 144, and a database 146. The API module 136 can store or persist all articles, recipes, programs, workouts and videos, and can deliver any or all of them when requested by another unit. The content engine 140 determines what should be displayed next on the application 123 to a particular user based on a list including display items identified in the order in which each item should be displayed. This list as well as the order is specific to each user, and the content unit 108 can prepare this list and order based on user's current health profile. When a user views a displayed item on the list, the list is updated to remove the already displayed items and only the remaining items remain on the list. This updating of the list can be performed using endpoints, description of which follows.

The content engine 140 can store (or persist) endpoints that can determine the specific content that has been viewed by a given user, and these endpoints can then mark that content as “read” for that particular user. Each endpoint can be a web service endpoint. Every endpoint can have a unique address. The endpoint address can be represented by the EndpointAddress class, which can contain a uniform resource identifier (URI) that can represent the address of the service (or the display item), an identity that represents the security identity of the service (or the display item), and a collection of optional headers. The optional headers can provide more detailed addressing information to identify or interact with the endpoint. For example, the headers can indicate how to process an incoming message, where the endpoint should send a reply message, or which instance of a service to use to process an incoming message from a particular user when multiple instances are available.

Each model 144 can be a collection of user-specific data that can identify the user uniquely, such as a username and/or password of that user. The model 144 can further store all biomarker data for that user. The model 144 can have a one to one mapping with tables that are associated with that user and are stored in the database 146. The one or more models 144 can facilitate the creation and use of business objects whose data requires persistent storage to a database 146. The one or more models 144 may only interact with the database 146.

The content unit 108 can store data that has been marked as “read” in a form that can be sent to any device that can output the data in a format readable or viewable by a user. For example, the admin module 142 can send the data to a computing server (not shown in FIG. 1) that can control the system 102. This computing server can use this data to add and manage existing data for the application 123. This data can be stored in the database 146. The content unit 108 can be a cluster of instances, such as EC2 instances. Each instance of the content unit 108 can be a virtual server, which can be scaled and deployed independently of the other units. Although the content unit 108 has been described as a virtual server, in an alternate implementation the content unit 108 can be a physical server, which can be a co-located server, an on-premise server, a collection of servers, and/or any other type of servers and/or any combination thereof. The one or more physical servers can be communicatively coupled using at least one of the following a wireless network, a wired network, a metropolitan area network, a wide area network, a local area network, a virtual local area network, and/or any other type of network and/or any combination thereof.

The account and identity unit 110 can be used to authenticate a user. The account and identity unit 110 can include an API module 148, one or more controllers 150, an admin module 152, models 154, and a database 155. The one or more controllers 150 can store each user's authentication data, such as a username and/or password, in the database 155. The one or more controllers 150 can store the authentication data using an adaptive cryptographic hash function for passwords, such as BYCRYPT, as a one-way hash as well as an encrypted unique user ID. The account and identity unit 110 may only be accessible to and called by the frontend unit 106. The account and identity unit 110 can be a cluster of instances, such as EC2 instances. Each instance of the account and identity unit 110 can be a virtual server, which can be scaled and deployed independently of the other units. Although the account and identity unit 110 has been described as a virtual server, in an alternate implementation the account and identity unit 110 can be a physical server, which can be a co-located server, an on-premise server, a collection of servers, and/or any other type of servers and/or any combination thereof. The one or more physical servers can be communicatively coupled using at least one of the following a wireless network, a wired network, a metropolitan area network, a wide area network, a local area network, a virtual local area network, and/or any other type of network and/or any combination thereof.

The secure health store unit 112 can include an API module 156, one or more controllers 158, an admin module 160, models 162, and a database 164. The one or more controllers 158 can store the following data for each user in the database 164: health information including the biomarkers, overall health score, and other metrics such weight or challenge data. Challenge data is data associated with corresponding one or more challenges for a user. The challenges can be specific behavioral goals assigned to individual users based on their biomarker data. For example, the system 102 may recommend to a user with a low activity score a challenge of, for example, walking 10,000 steps every day as a part of a goal program. All of the data can be keyed using the unique ID generated by the account and identity unit 110, but this is persisted using encryption, such as AES256 encryption. The API module 156 can provide an API to the frontend unit 106 that can allow the storage and retrieval of health data based on different time ranges. The secure health store unit 112 can be a cluster of instances, such as EC2 instances. Each instance of the secure health store unit 112 can be a virtual server, which can be scaled and deployed independently of the other units. Although the secure health store unit 112 has been described as a virtual server, in an alternate implementation the secure health store unit 112 can be a physical server, which can be a co-located server, an on-premise server, a collection of servers, and/or any other type of servers and/or any combination thereof. The one or more physical servers can be communicatively coupled using at least one of the following a wireless network, a wired network, a metropolitan area network, a wide area network, a local area network, a virtual local area network, and/or any other type of network and/or any combination thereof.

The notifications unit 114 can include an API module 166, one or more controllers 168, an admin module 170, models 172, and a database 174. The API module 166 can provide an API to the frontend unit 106. This API can allow the sending of communication data, scheduling of that communication data, or generating notifications. The communication data can include emails or text messages. In alternate implementations, the communication data can additionally or alternately include social network alerts and/or any other communication. The notifications can include push notifications or notifications via any other mode, such as short messaging service (SMS), email, social network notification, and/or the like. The API module 166 can integrate with notifications services, such as those provided by a third party. Some examples of third party services are SIMPLE EMAIL SERVICE by AMAZON, and APPLE PUSH NOTIFICATIONS. The notifications unit 114 can be a cluster of instances, such as EC2 instances. Each instance of the notifications unit 114 can be a virtual server, which can be scaled and deployed independently of the other units. Although the notifications unit 114 has been described as a virtual server, in an alternate implementation the notifications unit 114 can be a physical server, which can be a co-located server, an on-premise server, a collection of servers, and/or any other type of servers and/or any combination thereof. The one or more physical servers can be communicatively coupled using at least one of the following a wireless network, a wired network, a metropolitan area network, a wide area network, a local area network, a virtual local area network, and/or any other type of network and/or any combination thereof.

The integrations unit 116 can include an API module 166, one or more controllers 168, an admin module 170, models 172, and a database 174. The API module 166 can provide an API and customer integrations with third party services 120, such as phlebotomy providers, telomere lab results, and wearable device companies such as FITBIT and WITHINGS. The integrations unit 116 can be a cluster of instances, such as EC2 instances. Each instance of the integrations unit 116 can be a virtual server, which can be scaled and deployed independently of the other units. Although the integrations unit 116 has been described as a virtual server, in an alternate implementation the integrations unit 116 can be a physical server, which can be a co-located server, an on-premise server, a collection of servers, and/or any other type of servers and/or any combination thereof. The one or more physical servers can be communicatively coupled using at least one of the following a wireless network, a wired network, a metropolitan area network, a wide area network, a local area network, a virtual local area network, and/or any other type of network and/or any combination thereof.

The term unit, as used herein, can refer to one or more of: hardware components, software modules, and services. The user or individual can also be referred to as an entity, a client, and/or the like. The computing device 118 can be a laptop computer, a desktop computer, a tablet computer, a cellular smart phone, a phablet, a computing kiosk, and/or any other computing device. Any of the databases 134, 146, 156, 164, 174 and 184 can store data in a tabular format. One or more of those databases can be a hierarchical database. At least one of the databases 134, 146, 156, 164, 174 and 184 can be a columnar database, a row based database, or an in-memory database. The database 134, 146, 156, 164, 174 or 184 is an independent hardware entity when the corresponding unit is a software module or a service. Components in the backend 104 can communicate with those in the frontend 122 via a communication network, which can be a local area network, a wide area network, internet, intranet, Bluetooth network, infrared network, and/or other communication networks.

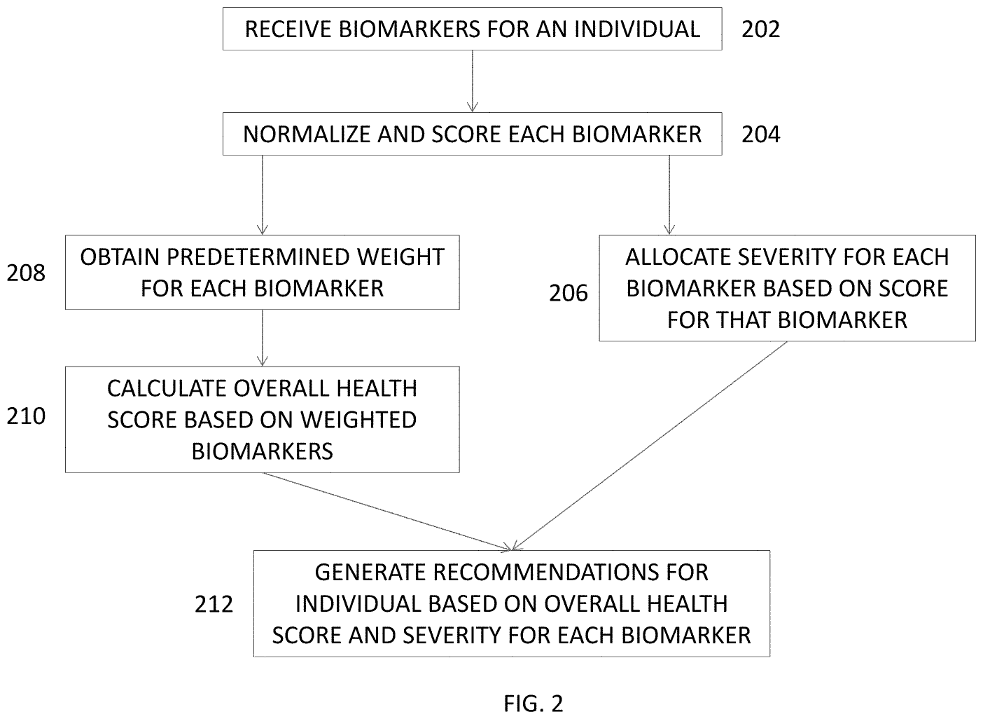

FIG. 2 is a flow diagram illustrating a calculation/computing/determination of health score for an individual based on biomarkers for that individual, and generation of recommendations for that individual based on severities calculated for those biomarkers. The API module 126 or the web module 128 of the frontend unit 106 can receive, at 202, health biomarkers for an individual or user from the application 123 on the computing device 118. The scoring module 130 can receive the biomarker data from one of the API module 126 and the web module 128. In an alternate implementation, the scoring module 130 may retrieve at least some biomarker data that is already stored in the database 134 from the database 134. The scoring module 130 can normalize and score, at 204, each biomarker. In one example, the score for each biomarker can range between zero and one hundred. The scoring module 130 can assign, at 206, severity to each biomarker. The severity for each biomarker can be healthy, mild, moderate and severe. For each biomarker in the aforementioned example, healthy can correspond to the score of 76-100, mild can correspond to the score of 51-75, moderate can correspond to the score of 26-50, and severe can correspond to the score of 0-25. In other implementations, any other suitable range for each of the following severities for each biomarker can be used: healthy, mild, moderate and severe. In another implementation, there can be any number of severities rather than four, as noted above.

The scoring module 130 can obtain, at 208, predetermined weight for each biomarker. The scoring module 130 can calculate, at 210, the overall health score for the individual based on weighted biomarkers. The content unit 108 can generate, at 212, recommendations for each individual based on overall health score, as calculated at 210, and severity for each biomarker based on a score for that biomarker, as calculated at 206. The recommendations can be treatment suggestions for the individual. The treatment suggestions can be behavioral changes recommended for the individual. In one example, mild hypertension treatment recommendations may be less severe than recommendations for severe hypertension. The specific content of the treatment recommendations (for example, short messaging service (SMS) text and videos) can be driven by the individual's biomarker scores, and can be specific to the user's unique combination of those biomarker scores. This generates a highly individualized collection of behavioral recommendations that are: specific to disease and severity, generated instantly, and made available constantly (for example, twenty four hours a day, seven days a week, and every day of the year) on the application 123 at the computing devices 118. The instant generation of the recommendations refers to the generation of those recommendations immediately after the one or more values for the one or more biomarkers is received “Immediately” or “immediately after” can refer to a time gap of up to 0.1 second. In an alternate implementation, this time gap can be up to 1 second. In a yet another implementation, this time gap can be up to 5 seconds. In an alternately implementation, this time gap can be up to 20 seconds or more.

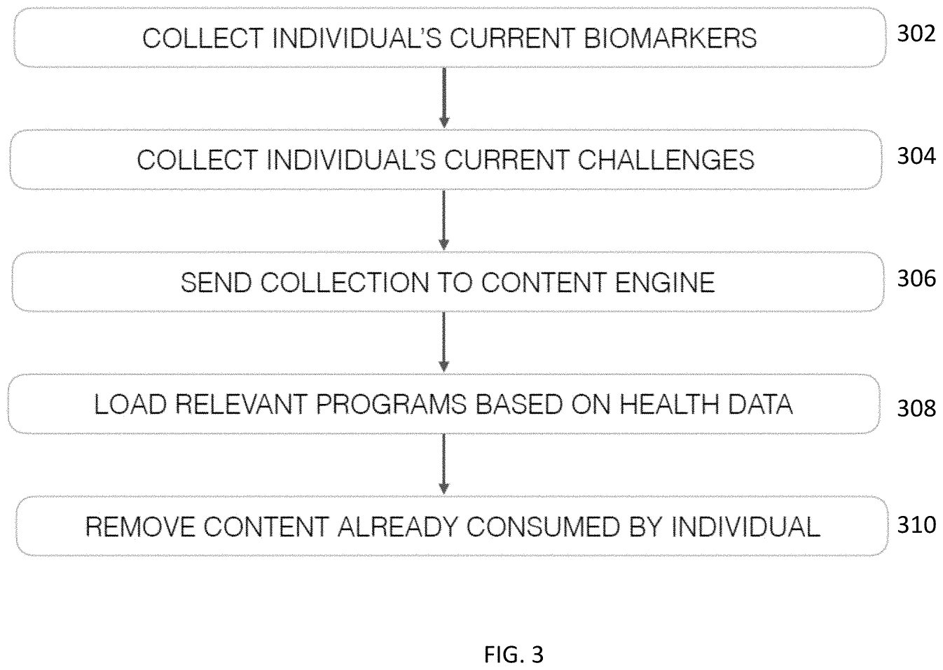

FIG. 3 is a flow diagram illustrating a collection and storage of current values for biomarkers of an individual. The application 123 can collect (for example, receive as input), at 302, current values of biomarkers for an individual or user. The application 123 can collect (for example, receive as input), at 304, current values of challenges for an individual or user. The application 123 can send, at 306, the collected values of biomarkers and challenges for the user to the content engine 140 via the frontend unit 106. The content engine 140 can store the received data in the database 146. The one or more controllers 138 can load, at 308, relevant programs based on the health data. The one or more controllers 138 can remove, at 310, the content already consumed by the user from the database 146.

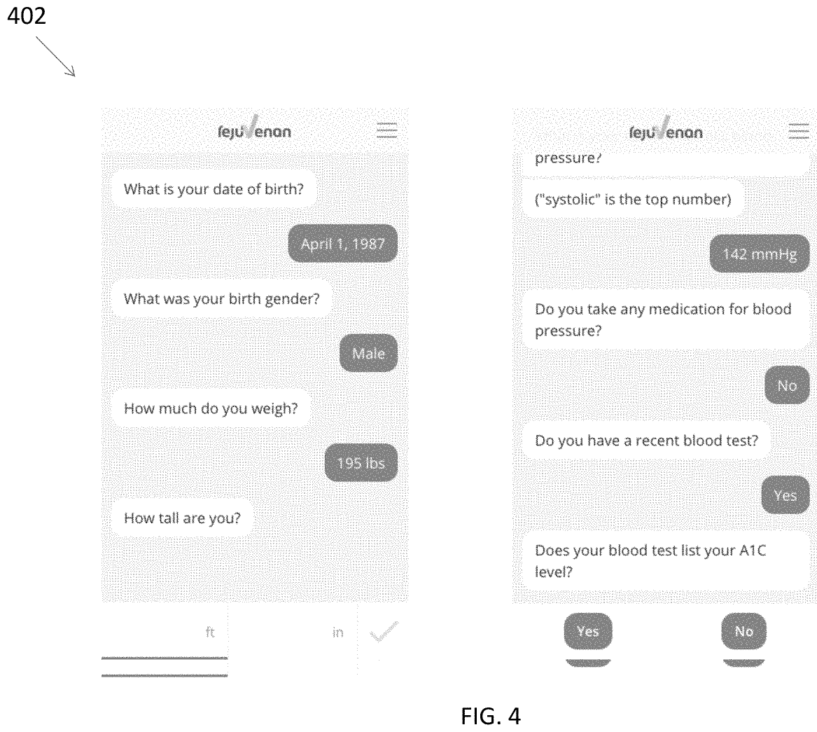

FIG. 4 illustrates a screenshot 402 of the application 123 where the user can input data to receive a health score. The screenshot 402 shows the biomarkers of birthday, birth gender, and weight. While the shown example shows these simple biomarkers, the biomarkers can be more complicated in other examples. In general, a biomarker here can refer to any measurement related to the biological system of an individual being assessed and/or treated, as noted above. The screenshot 402 shows a back and forth between the automated application 102 and the user, which can happen in real-time.

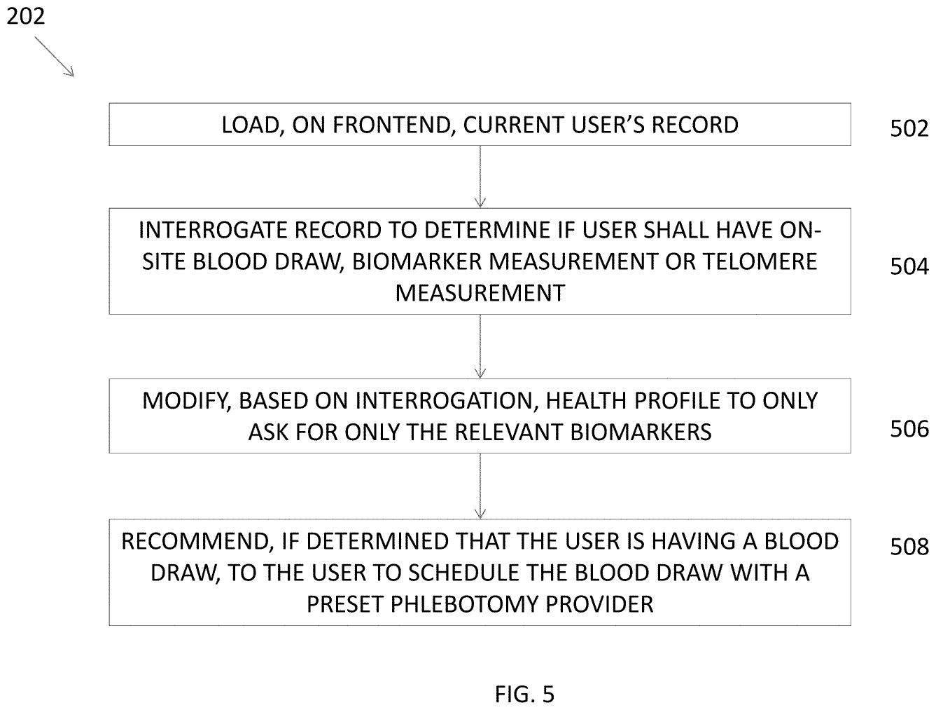

FIG. 5 is a flow diagram illustrating the process 202 of a selection of biomarkers, values for which are interrogated from the user. The frontend unit 106 can load, at 502, the current user's record including biomarker data and challenge data of that user. The one or more controllers 124 can interrogate, at 504 and using Boolean logic, this record to determine if the user will have an on-site blood draw, biomarker measurement or telomere measurement. Based on this result, the one or more controllers 124 can modify, at 506, the health profile such that the application 123 only asks for the relevant biomarkers. If the user is having a blood draw, the application 123 can generate an automated recommendation of scheduling the blood draw with a preset phlebotomy provider. In other implementations, any other biomarker may be used. In some implementations, the application 123 can recommend and/or schedule a clinician's visit automatically based on the biomarker values received from the user. The clinician referred herein can be a doctor, a nurse, a laboratory personnel, a physiotherapist, or any other medical personnel.

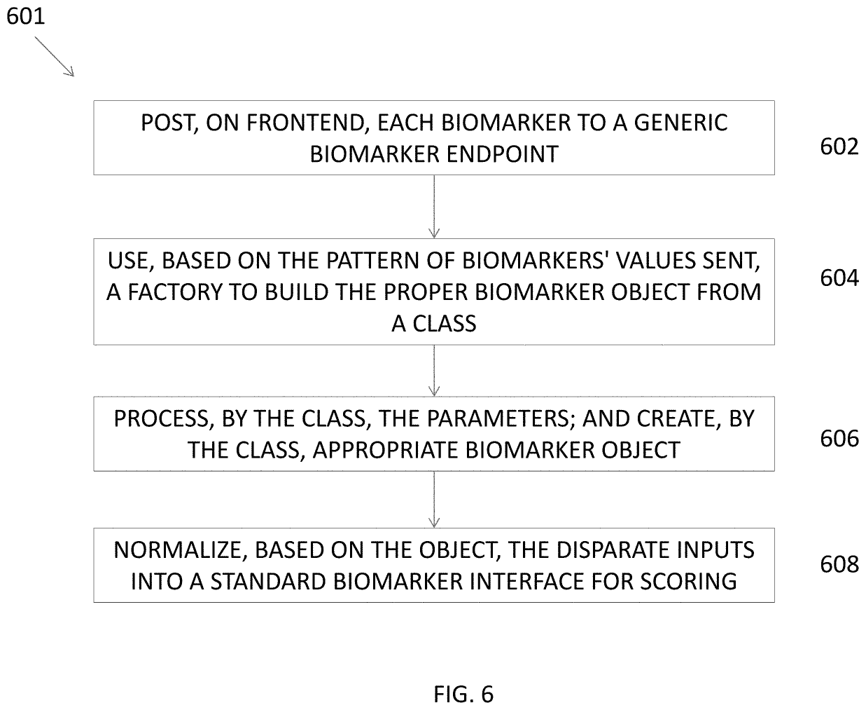

FIG. 6 is a flow diagram illustrating the sub-process 601 of normalization of each biomarker within the process 204. The frontend unit 106 can post, at 602, data characterizing a biomarker to a generic biomarker endpoint. In one example, this posting can be a RESTful POST to an API endpoint. The frontend unit 106 can use, at 604 and based on the pattern of biomarkers' values sent, a programming factory to build a proper biomarker programming object from a programming class. The programming factory can be a programming function for generating programming objects. The one or more controllers 158 can process, at 606, the parameters and creates appropriate biomarker object. The frontend unit 106 can normalize, at 608 and based on the object, the disparate inputs into a standard biomarker interface for scoring.

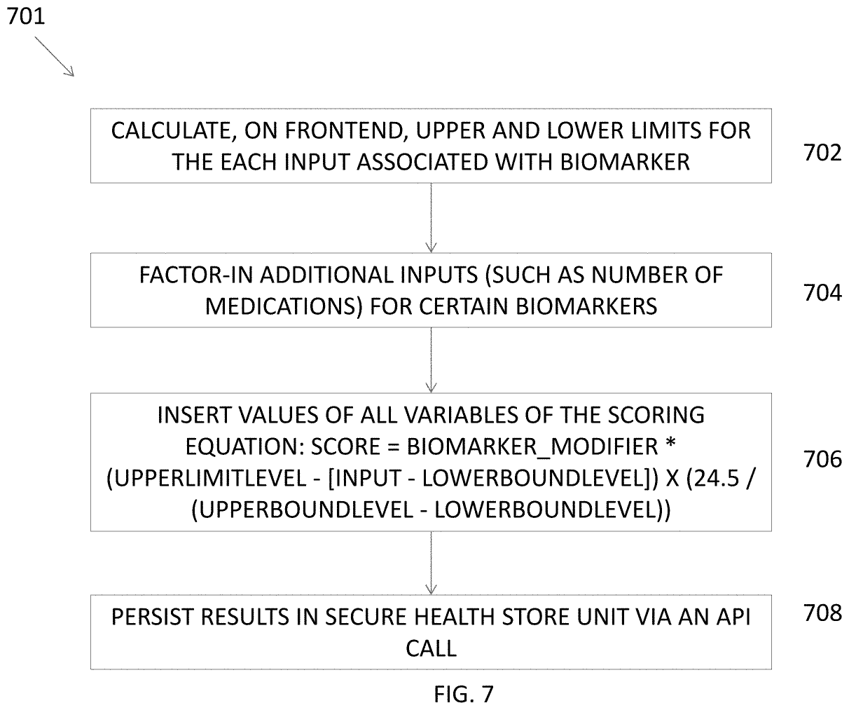

FIG. 7 is a flow diagram illustrating the sub-process 701 of scoring each normalized biomarker within the process 204. The frontend unit 106 can calculate, at 702, upper and lower bounds for each value input by the user for each biomarker. In one example, the biomarker A1C may be associated with input values of A1C and medications taken by the user to obviate any problem due to the A1C levels of the user. Thus, each biomarker may be associated with one or more input values. In another example, the biomarker can be alcohol, and the values input for alcohol may be quantity of alcohol consumed by the user each time period (for example, every day). The frontend unit 106 can factor in (that is, account for), at 704 and for certain biomarkers, additional inputs, such as number of medications. The scoring module 130 plugs in values of all variables into the scoring equation: score=biomarker_modifier*(UpperLimitLevel−[Input−LowerBoundLevel])×(variable/(UpperBoundLevel−LowerBoundLevel)). In one implementation, the “variable” in this equation can have a constant value of 24.5. In another implementation, the “variable” can have any constant value between 1 and 40. In yet another alternate implementation, the “variable” can have any possible numeric value. The scoring module 130 can send the score for each biomarker of an individual to the secure health store unit 112 via an API call. The secure health store unit 112 can persist the score at 708.

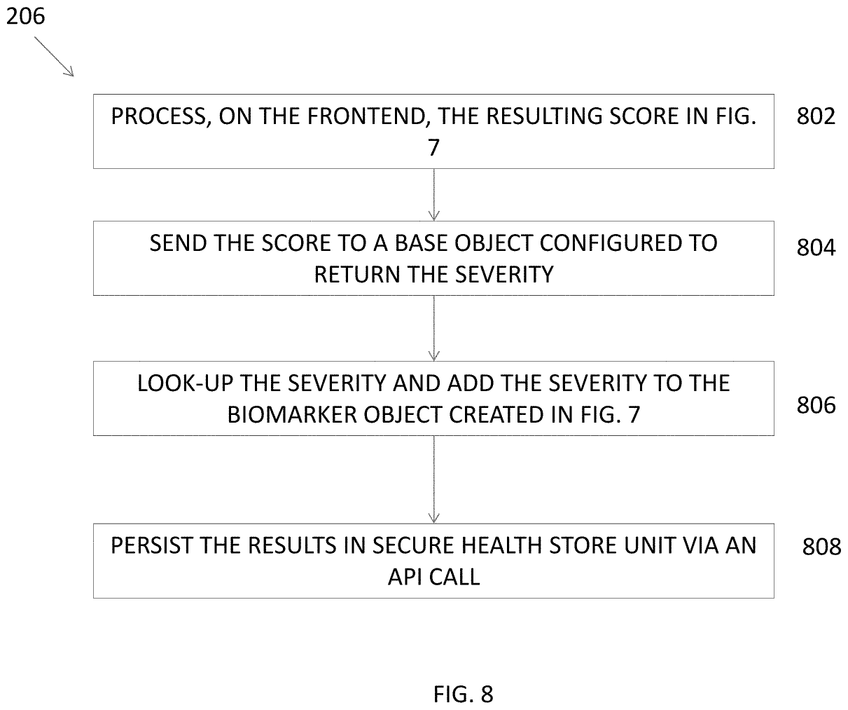

FIG. 8 is a flow diagram illustrating the process 206 of assigning severity to each normalized biomarker. The severity for each biomarker can be healthy, mild, moderate and severe. For each biomarker in the aforementioned example, healthy can correspond to the biomarker score of 76-100, mild can correspond to the biomarker score of 51-75, moderate can correspond to the biomarker score of 26-50, and severe can correspond to the biomarker score of 0-25. In other implementations, any other suitable range for each of the following severities for each biomarker can be used: healthy, mild, moderate and severe. In another implementation, there can be any number of severities rather than four, as noted above.

The frontend unit 106 can process, at 802, the score as described by FIG. 7. The frontend unit 106 can then send, at 804, the score to a base object to return the severity. The aforementioned base object can be a base object in an object oriented design. The frontend unit 106 can then look up, at 806 using the objects stored in the frontend unit 106, the severity to the biomarker object created in FIG. 7. The scoring module 130 can send the severity to the secure health store unit 112 via an API call. The secure health store unit 112 can persist the severity at 808.

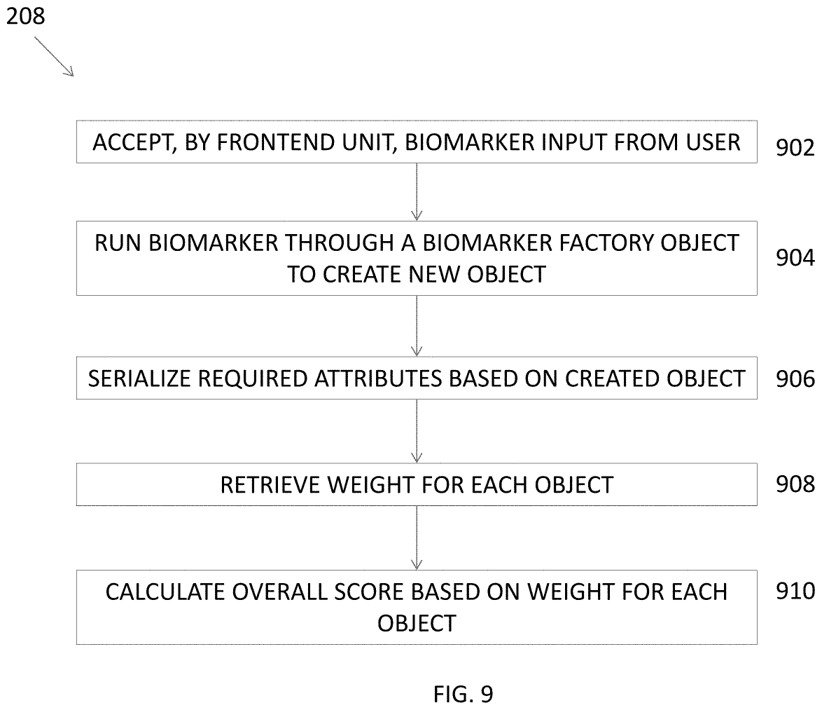

FIG. 9 is a flow diagram illustrating the process 208 of obtaining the predetermined weight for each biomarker, and using the weighted biomarkers for computing the overall score. The frontend unit 106 can receive, at 902, the value of the biomarker input by the user on the application 123. The frontend unit 106 can run the biomarker through, at 904, a biomarker programming factory object, which creates a new object. The running through of the biomarker can refer to the processing or building of the biomarker. Based on the new object, the frontend unit 906 can serialize the required attributes. Serialization can be the process of translating data structures or object state into a format that can be stored and reconstructed later in the same or another computer environment. The frontend unit 106 can retrieve, at 908, a weight for each finalized biomarker object. The frontend unit 106 can calculate, at 910, an overall score the user based on the weights for the biomarker objects.

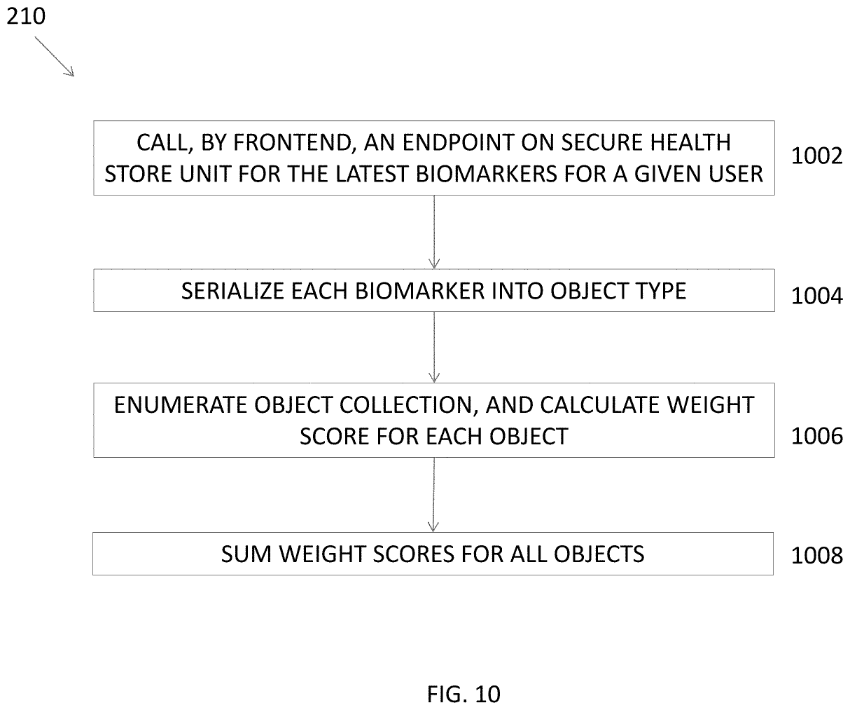

FIG. 10 is a flow diagram illustrating the process 210 of calculating the health score. The frontend unit 106 can call, at 1002, an endpoint on the secure health store unit 112 for the most recent biomarkers for a particular user. The frontend unit 106 can serialize, at 1004, each biomarker into its proper object type. The frontend unit 106 can enumerate, at 1006, the object collection, where each object is associated with its weight score. For each biomarker object, the weighted score can be equal to a multiplied produce of the score for that biomarker and the weight computed for that biomarker. The scoring module 130 can sum (that is, add), at 1008, the weight scores.

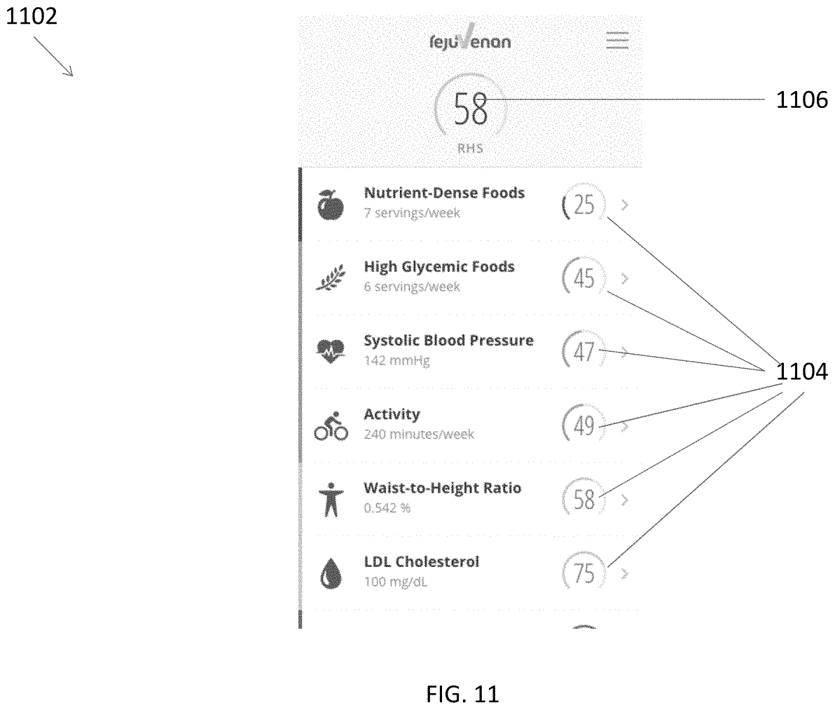

FIG. 11 illustrates a screenshot 1102 of the application 123 showing the health score 1104 of each biomarker for a user as well as the combined health score 1106. The score 1104 for each biomarker can represent severity, based on the current medical literature or research, of a particular disease or physiological condition known to be associated with a particular biomarker (for example, the serum A1C biomarker is associated with diabetes). The score 1106 can represent an assessment of the overall health and wellness of an individual. In some implementations, a higher score 1106 can indicate better health.

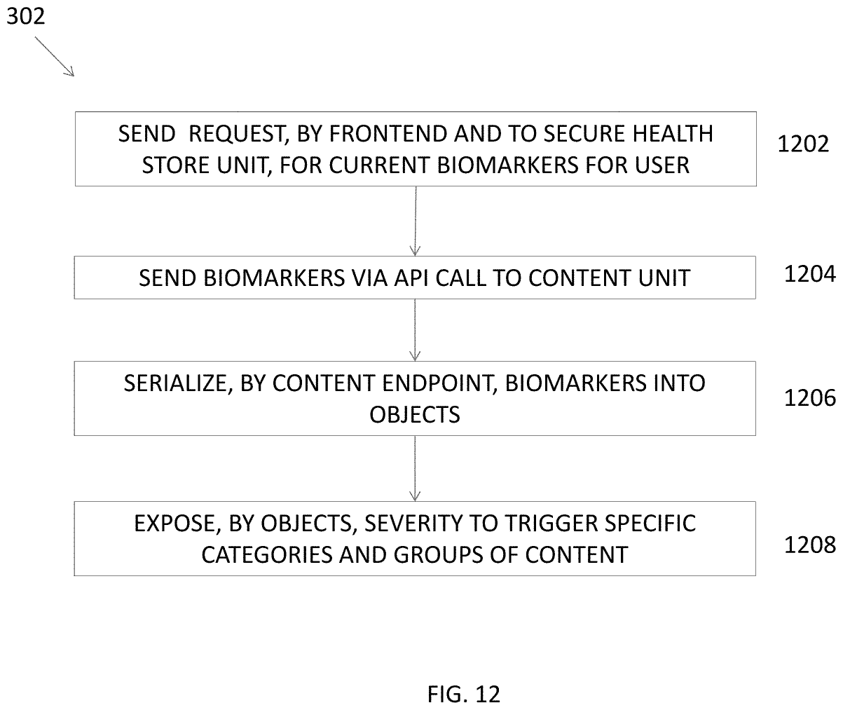

FIG. 12 is a flow diagram illustrating the process 302 of collecting the current biomarkers for a user. The frontend unit 106 can make, at 1202, a call to the secure health store unit 112 to collect the current biomarkers for a given user. The frontend unit 106 can send, at 1204, the biomarkers to the content unit 108 via an API call. The content endpoint, which can be a web service endpoint (for example, a RESTful API endpoint), within the content unit 108 can serialize, at 1206, the biomarker into programming objects. The content unit 108 can enable, at 1208, the objects to expose (for example, output) the severity (for example, one of healthy, mild, moderate and severe) of a biomarker of the user so that the system 102 can recommend customized treatments to the user. The system 102 can recommend treatments by retrieving and displaying content associated with those treatments.

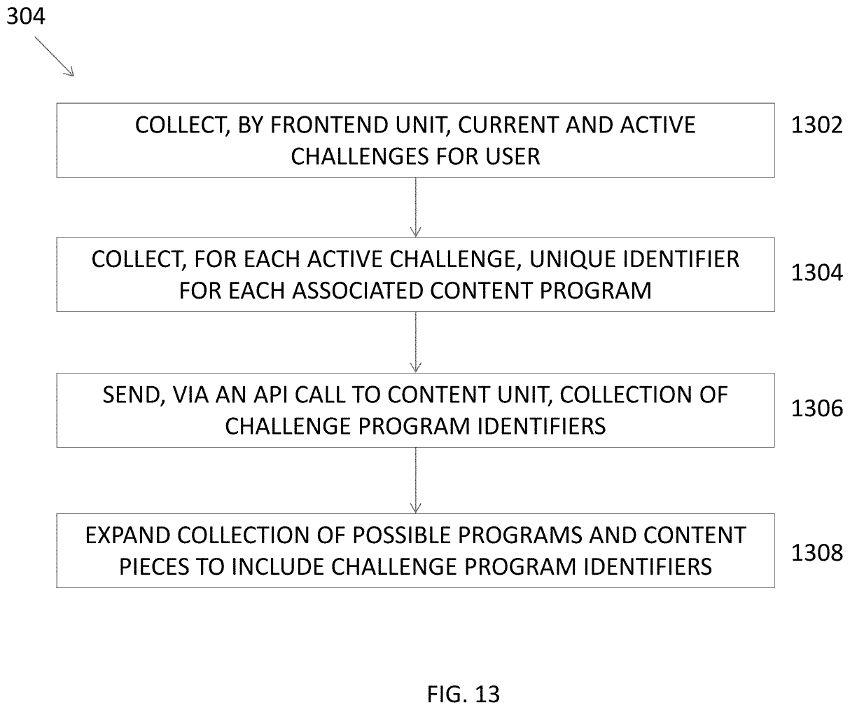

FIG. 13 is a flow diagram illustrating the process 304 of collecting the current challenges faced by a user. The frontend 106 can collect, at 1302, the current active challenges for a current user. For each active challenge, the frontend 106 can collect, at 1304, the unique identifier for each associated content program (for example, an identifier uniquely identifying either a corresponding challenge program of the user). The frontend 106 can send, at 1306, the collection of challenge program identifiers to the content unit 108 via an API call. The content unit 108 can expand, at 1308, the collection of possible programs and child content pieces to include these challenge program identifiers. The content unit 108 can perform this expansion by using SQL joins with primary and foreign keys.

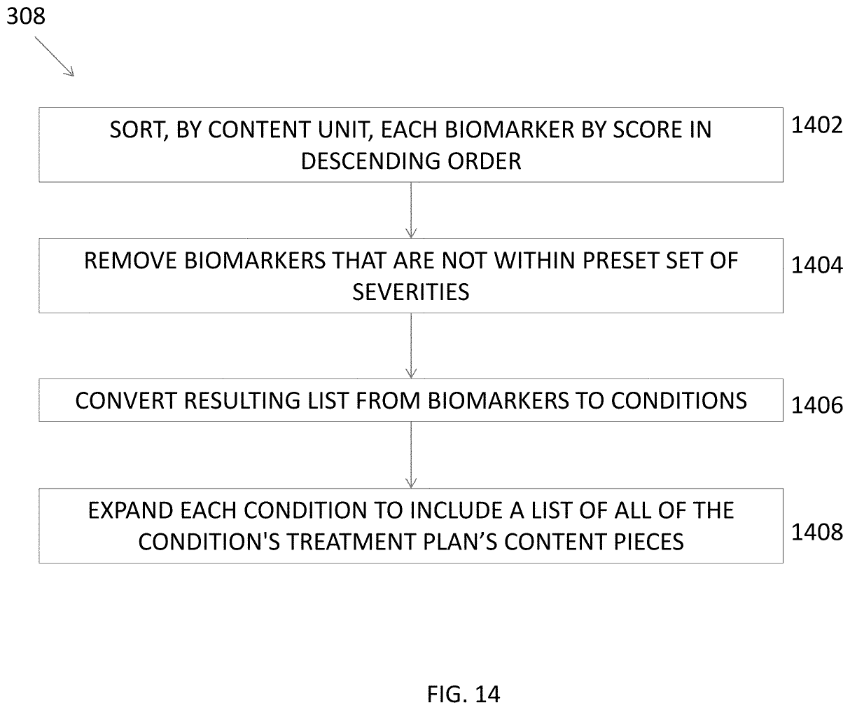

FIG. 14 is a flow diagram illustrating the process 308 of loading of relevant programs based on health data. The content unit 108 can sort, at 1402, each biomarker by score in descending order. The content unit 108 can remove, at 1404, any biomarker that is not within the given set of severities (for example, healthy, mild, moderate, and severe). The content unit 108 can convert, at 1406, the resulting set from biomarkers to conditions. The conditions can refer to one or more of the following: obesity, inadequate physical activity, diabetes, cardiovascular disease, hypertension, excess alcohol intake, smoking, and any combination thereof. The content unit 108 can expand, at 1408, data associated with each condition to include a list of the entire content (for example, recommended videos and behavioral suggestions) associated with that condition's treatment plan.

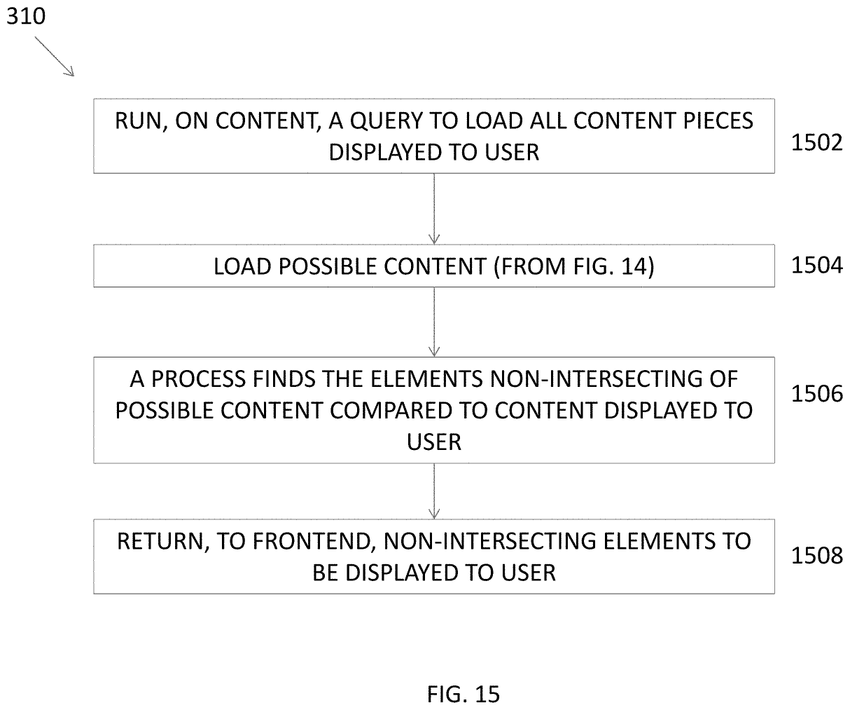

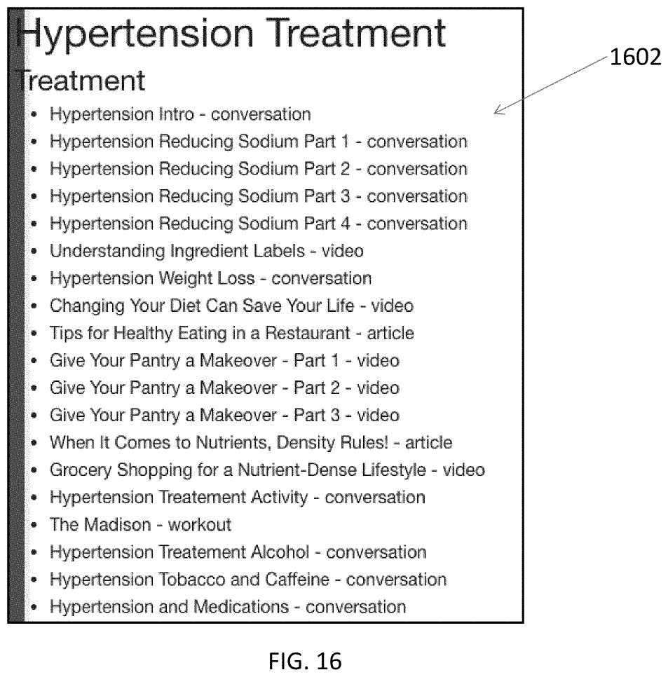

FIG. 15 is a flow diagram illustrating the process 310 of the removal of the content already displayed to the user. The content unit 108 can run, at 1502, a query to load all content pieces displayed to, or interacted with by, the current user. The content unit 108 can load, at 1504, the set of possible content as generated in FIG. 14. The content unit 108 can execute, at 1506, a process that can find the elements non-intersecting of content loaded in 1502 compared to content loaded in 1504. Computationally, the step of 1506 can be performed by a query with an exclusion predicate to identify elements (referred to as non-intersecting elements) that do not have a given set of foreign keys. The content unit 108 can send, at 1508, the result of 1506 to the frontend unit 106. The content that has already been displayed to the user can be removed. This removal of content is further clarified by FIG. 16.

FIG. 16 illustrates a list of content pieces 1602 arranged in an order in which they are displayed to a user. The content piece 1602 that has already been displayed to a user is removed after it has been displayed and the user has interacted with it, if such an interaction is required or deemed important.

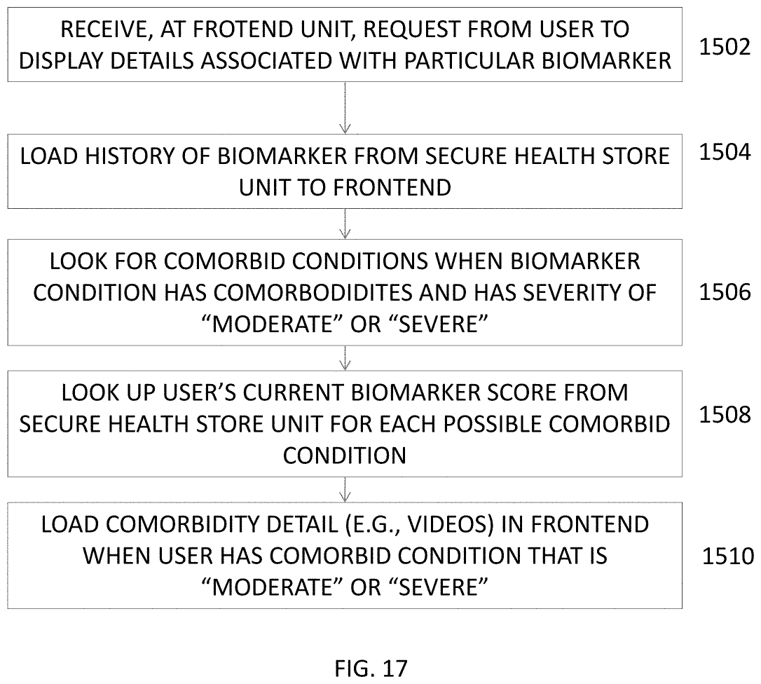

FIG. 17 is a flow diagram illustrating a display of comorbidities. The frontend unit 106 can receive, at 1502, a request from a user to display details associated with particular biomarker. The frontend unit 106 can load, at 1504, history of the biomarker data from the secure health store unit 112. The frontend unit 106 can look, at 1506, for comorbid conditions when biomarker condition has comorbidities and has severity of “moderate” or “severe.” The frontend unit 106 can look up, at 1508, user's current biomarker score from secure health store unit 112 for each possible comorbid condition. The frontend unit 106 can load, at 1510, comorbidity detail (for example, videos and/or pictures) in the frontend unit 106 when the user has a comorbid condition that is “moderate” or “severe.”

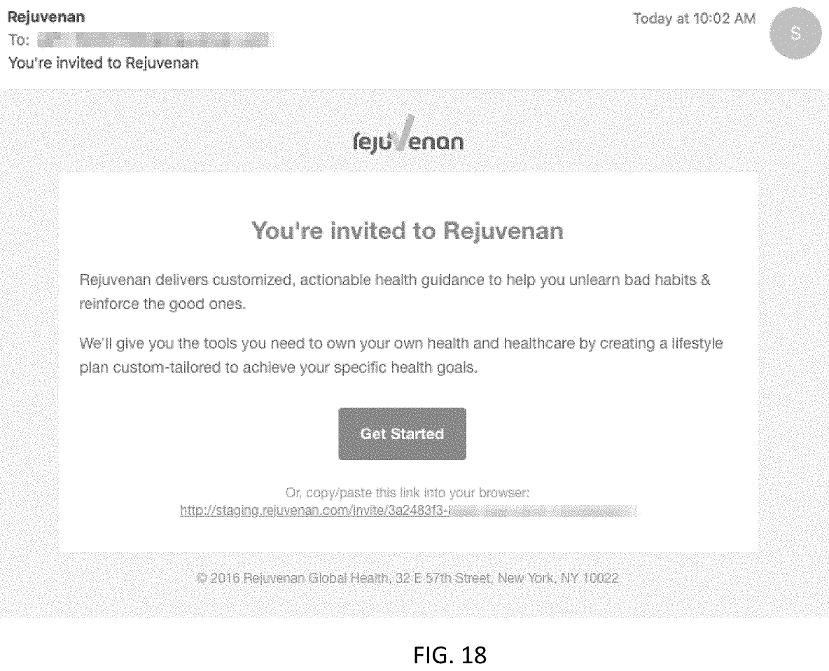

FIG. 18 illustrates a graphical user interface displaying an email invitation for a user as sent by the system 102. The email invitation can be generated and sent by the notifications unit 114.

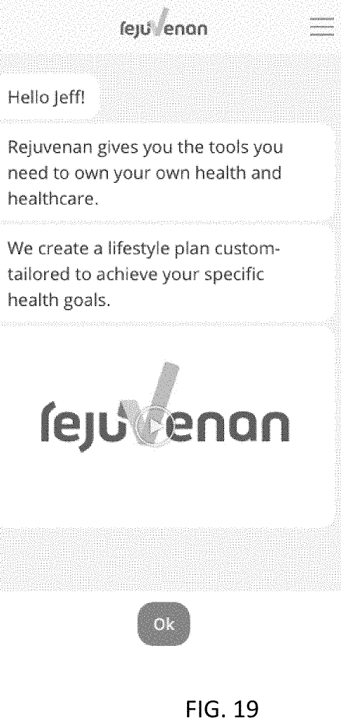

FIGS. 19-33 illustrate graphical user interfaces displayed by the application 123, as noted in greater detail below.

FIG. 19 illustrates a graphical user interface of the application 123 displaying an overview of the application 123.

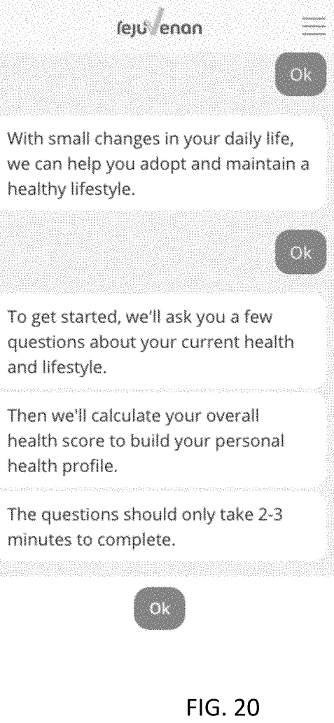

FIG. 20 illustrates a graphical user interface of the application 123 displaying further overview of the application 123.

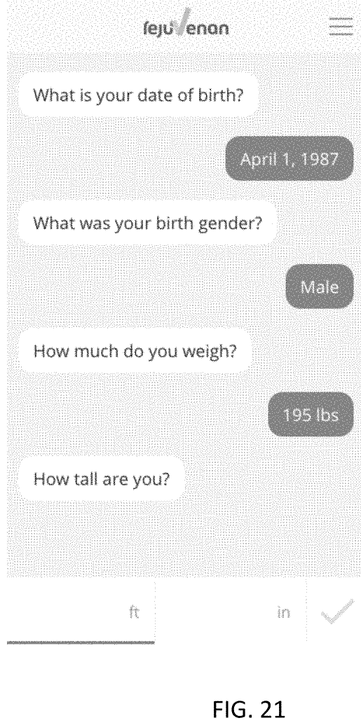

FIG. 21 illustrates a graphical user interface of the application 123 displaying receipt of values of biomarkers to create a health profile of a user. The values of the biomarkers can be received by the frontend unit 106 from the application 123. The values of the biomarkers can then be stored in the secure health store unit 112.

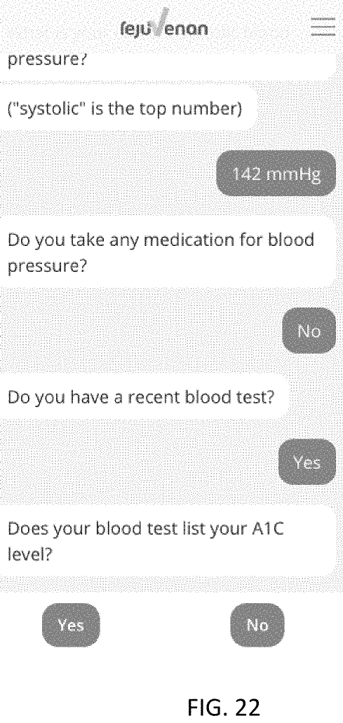

FIG. 22 illustrates another graphical user interface of the application 123 displaying receipt of values of more biomarkers to create a health profile of a user. The values of the biomarkers can be received by the frontend unit 106 from the application 123. The values of the biomarkers can then be stored in the secure health store unit 112.

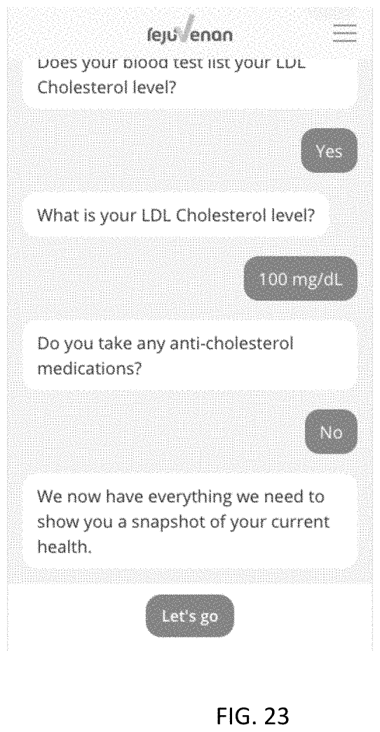

FIG. 23 illustrates another graphical user interface of the application 123 displaying receipt of values of more biomarkers to create a health profile of a user. The values of the biomarkers can be received by the frontend unit 106 from the application 123. The values of the biomarkers can then be stored in the secure health store unit 112.

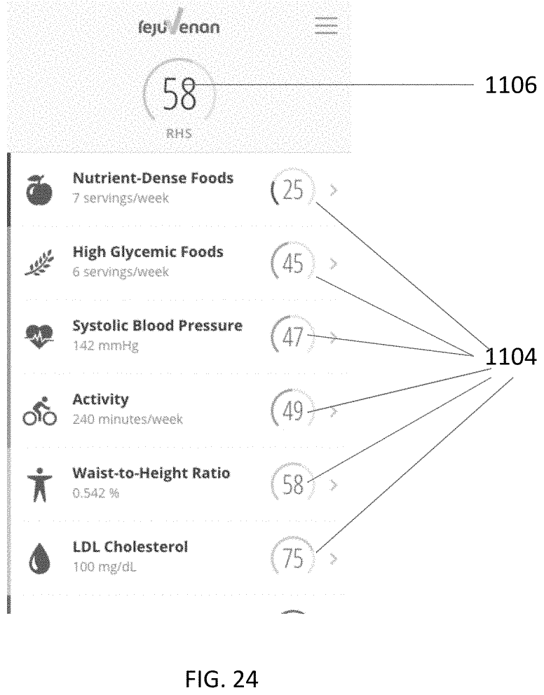

FIG. 24 illustrates a graphical user interface of the application 123 displaying biomarker scores 1104 for the user and an overall health score 1106 for that user. The biomarker scores 1104 can be sent to the application 123 by the frontend unit 106.

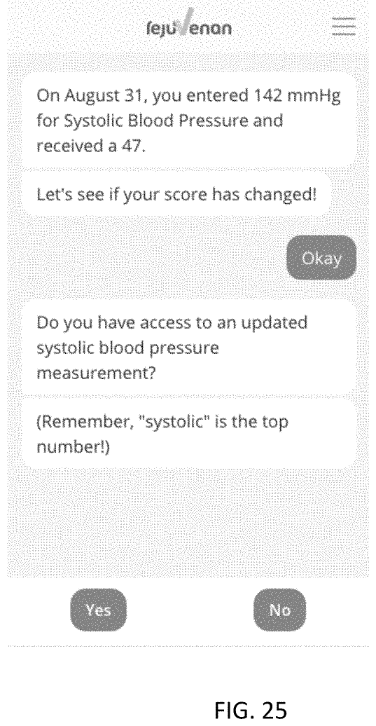

FIG. 25 illustrates a graphical user interface of the application 123 displaying an interactive update for data associated with each biomarker. This interactive update can also display recommendations to educate the user.

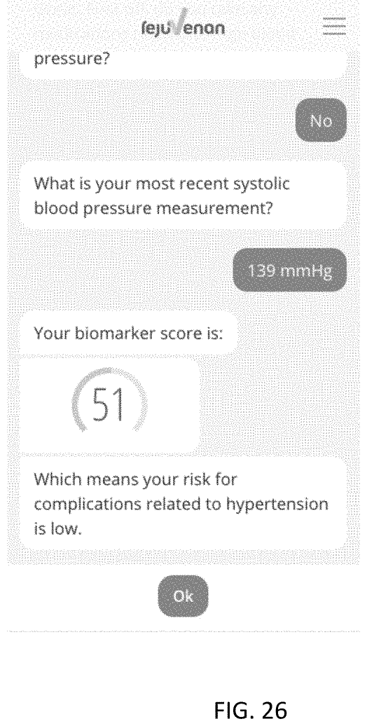

FIG. 26 illustrates another graphical user interface of the application 123 displaying another interactive update for data associated with each biomarker. This interactive update can also display one or more recommendations and/or one or more lessons/points to educate the user. In one implementation, such recommendations can be generated by the frontend unit 106 by using the data stored in the content unit 108.

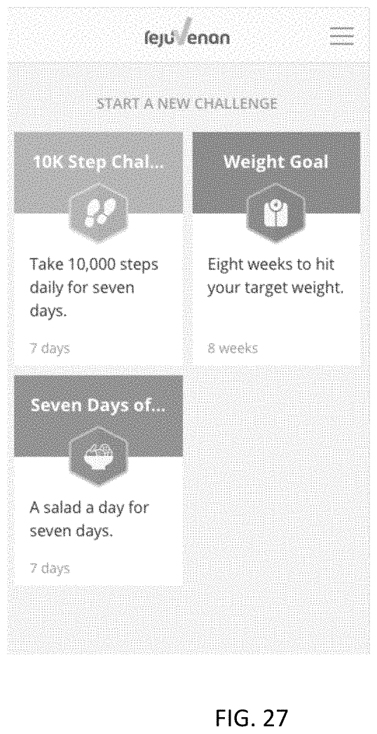

FIG. 27 illustrates a graphical user interface of the application 123 displaying challenge programs (which can also be referred to as challenge content or challenge data) recommended for the user based on the user's biomarker scores 1104 and the overall score 1106. The challenge content can be stored in the secure health store unit 112, and can be retrieved from there by the frontend unit 106, which can then send the challenge content to the application 123 for display on the computing device 118.

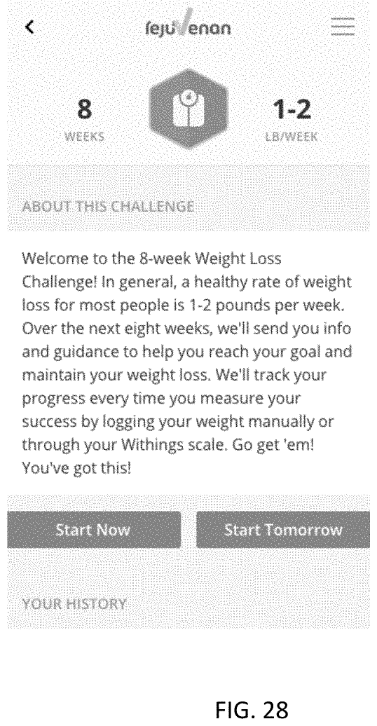

FIG. 28 illustrates a graphical user interface of the application 123 displaying details of a challenge program selected by the user. The details of the challenge program can be stored in the secure health store unit 112, and can be retrieved from there by the frontend unit 106, which can then send those details to the application 123 for display on the computing device 118.

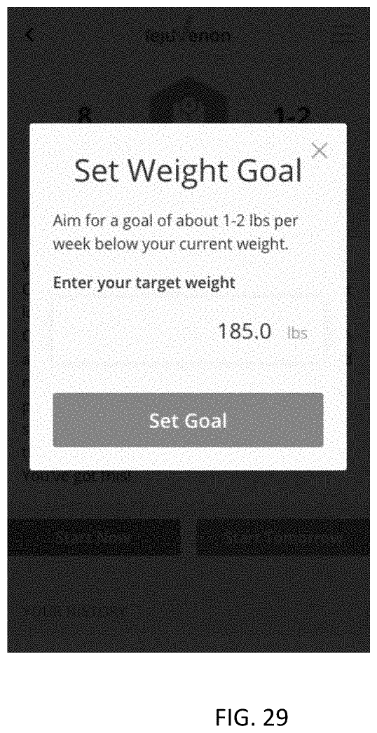

FIG. 29 illustrates a graphical user interface of the application 123 displaying details of a challenge program selected by the user. In the shown example, the challenge program is a program to reduce weight. The details of this challenge program can be stored in the secure health store unit 112, and can be retrieved from there by the frontend unit 106, which can then send those details to the application 123 for display on the computing device 118.

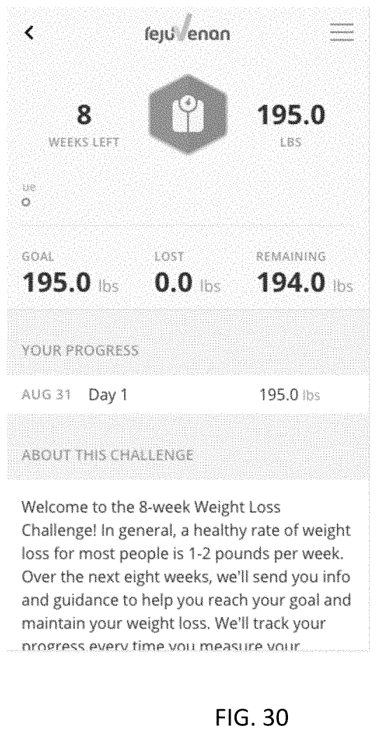

FIG. 30 illustrates a graphical user interface of the application 123 displaying further details of the weight challenge program described by FIG. 29. The details of the weight challenge program can be stored in the secure health store unit 112, and can be retrieved from there by the frontend unit 106, which can then send those details to the application 123 for display on the computing device 118.

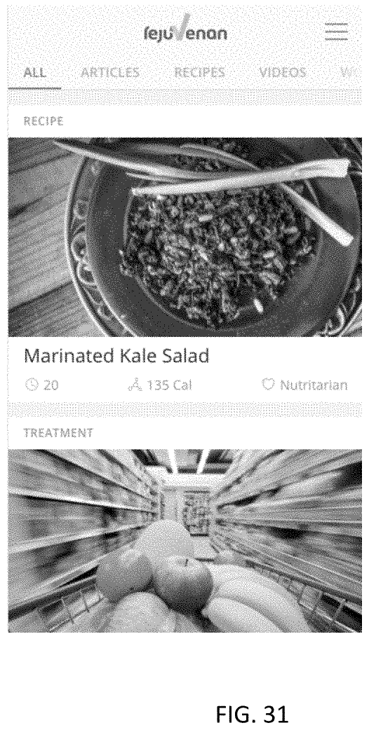

FIG. 31 illustrates a graphical user interface of the application 123 displaying a portion of a library of articles, recipes, videos, pictures, and any other data that are stored in the system 102 and made available to a user. The data characterizing the library can be stored within the content unit 108, and the data therein can be retrieved by the frontend unit 106 as and when required.

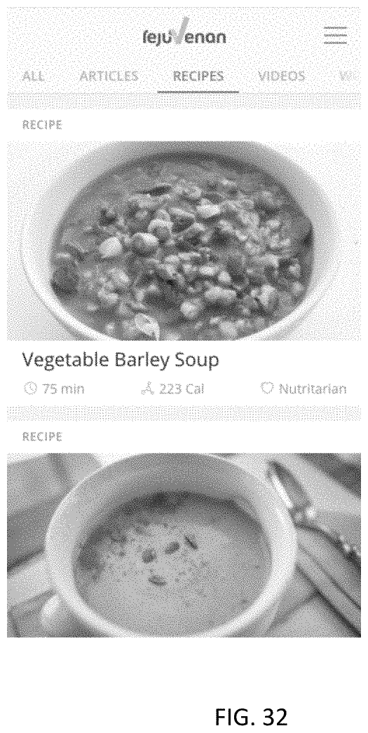

FIG. 32 illustrates a graphical user interface of the application 123 displaying another portion of the library of articles, recipes, videos, pictures, and any other data that are stored in the system 102 and made available to a user. The data characterizing the library can be stored within the content unit 108, and the data therein can be retrieved by the frontend unit 106 as and when required.

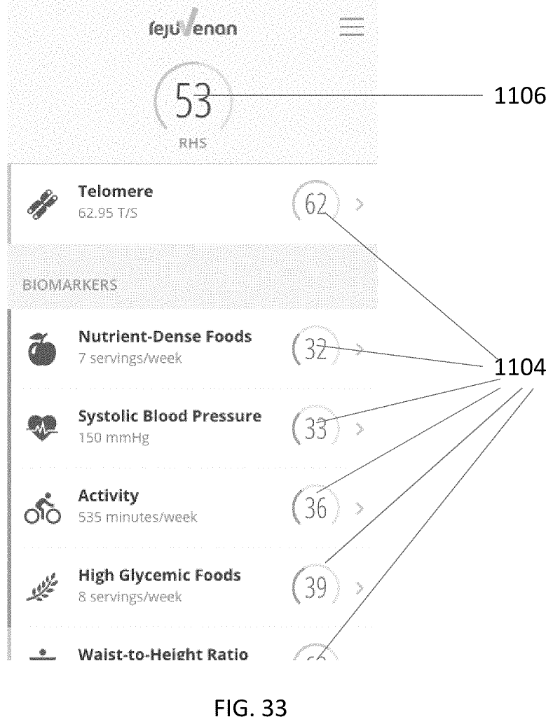

FIG. 33 illustrates another graphical user interface of the application 123 displaying biomarker scores 1104 for the user and an overall health score 1106 for that user. The biomarker scores 1104 and the overall health score 1106 can be a part of the health profile of the user. The biomarker scores 1104 can be sent to the application 123 by the frontend unit 106.

### Methods for Determining, Treating, or Reducing the Likelihood of Developing One or More Physiological Conditions

The apparatuses, systems, methods, non-transitory computer programmable products, and/or articles described herein identify and aid individuals who have developed one or more physiological conditions (such as, chronic diseases) or who are at risk of developing one or more physiological conditions to prevent or reduce the occurrence of the condition by providing the individual with instructions for enacting specific and customized lifestyle changes that match the an individual's biochemical, genetic, medical, and/or behavioral biomarker profile which is determined based an overall health score computed based on the input of one or more biomarkers, as described herein. Improvements in an individual's lifestyle are monitored by repeatedly measuring the changes in the one or more biomarkers. The individual continues to be monitored until such time as the individual's overall health score indicates an absence of one or more physiological conditions.

### Determining if an Individual has a Physiological Condition

Provided herein are methods for determining if an individual has one or more physiological conditions. The method involves receiving, by one or more processors, a score for each biomarker of one or more biomarkers for an individual and a severity for each biomarker, the severity being one of healthy, mild, moderate or severe; retaining, by the one or more processors, at least one biomarker within the one or more biomarkers that corresponds to a predetermined set of severities, the retaining encompassing removing, by the one or more processors, of at least one biomarker within the one or more biomarkers that neither has a severity assigned to it nor has a severity that does not fall within the predetermined set of severities; and determining, by the one or more processors, a physiological condition associated with the score of each retained biomarker based on the severity of the biomarker score. The physiological condition can be one or more of cardiovascular disease, diabetes, excessive alcohol consumption, hypertension, poor nutrition, obesity, and/or smoking. Each biomarker is assigned a severity score that ranges from 1-100.

The predetermined set of severities for determining if an individual has a particular physiological condition (for example, cardiovascular disease, diabetes, excessive alcohol consumption, hypertension, poor nutrition, obesity, and/or smoking) is mild, moderate or severe.

In some embodiments, the individual is determined to have mild cardiovascular disease if the individual has an LDL cholesterol biomarker score of between about 51-75, the individual is determined to have moderate cardiovascular disease if the individual has an LDL cholesterol biomarker score of between about 26-50 and severe cardiovascular disease if the individual has an LDL cholesterol biomarker score of between about 0-25. The individual is determined to have mild diabetes if the individual has an serum A1C biomarker score of between about 51-75, moderate diabetes if the individual has a serum A1C biomarker score of between about 26-50 and severe diabetes if the individual has an serum A1C biomarker score of between about 0-25. The individual is determined to have mild excessive alcohol consumption if the individual has an alcohol biomarker score of between about 51-75, moderate excessive alcohol consumption if the individual has an alcohol biomarker score of between about 26-50 and severe excessive alcohol consumption if the individual has an alcohol biomarker score of between about 0-25. The individual is determined to have mild hypertension if the individual has a systolic blood pressure biomarker score of between about 51-75, moderate hypertension if the individual has a systolic blood pressure biomarker score of between about 26-50 and severe hypertension if the individual has a systolic blood pressure biomarker score of between about 0-25. The individual is determined to have mild poor nutrition if the individual has a glycemic food intake or nutrient dense food intake biomarker score of between about 51-75, moderate poor nutrition if the individual has a glycemic food intake or nutrient dense food intake biomarker score of between about 26-50 and severe poor nutrition if the individual has a glycemic food intake or nutrient dense food intake biomarker score of between about 0-25. The individual is determined to have mild obesity if the individual has a waist to height ratio biomarker score of between about 51-75, moderate obesity if the individual has a waist to height ratio biomarker score of between about 26-50 and severe obesity if the individual has a waist to height ratio biomarker score of between about 0-25. The individual is determined to have mild smoking risk if the individual has a smoking biomarker score of between about 51-75, moderate smoking risk if the individual has a smoking biomarker score of between about 26-50 and severe smoking risk if the individual has an smoking biomarker score of between about 0-25.

### Reducing the Likelihood of Developing a Physiological Condition

Also provided herein is a method for reducing a likelihood of developing one or more physiological conditions. The method involves receiving, by one or more processors, a score for each biomarker of one or more biomarkers for an individual and a severity for each biomarker, the severity being one of healthy, mild, moderate or severe; retaining, by the one or more processors, at least one biomarker within the one or more biomarkers that corresponds to a predetermined set of severities, the retaining encompassing removing, by the one or more processors, of at least one biomarker within the one or more biomarkers that neither has a severity assigned to it nor has a severity that does not fall within the predetermined set of severities; determining, by the one or more processors, a physiological condition associated with the score of each retained biomarker; determining, by the one or more processors, a recommendation for improving the physiological condition; and sending, by the one or more processors, the recommendation to a computing application, the recommendation being used to reduce the likelihood of the individual developing one or more physiological conditions.

The physiological condition can be one or more of cardiovascular disease, diabetes, excessive alcohol consumption, hypertension, poor nutrition, obesity, and/or smoking. Each biomarker is assigned a severity score that ranges from 1-100.

The predetermined set of severities for reducing the likelihood that an individual will develop one or more physiological conditions (for example, cardiovascular disease, diabetes, excessive alcohol consumption, hypertension, poor nutrition, obesity, and/or smoking) are mild, moderate, and/or severe. Specifically, in some embodiments, the individual is determined to have mild risk of cardiovascular disease if the individual has an LDL cholesterol biomarker score of between about 51-75, moderate cardiovascular disease if the individual has an LDL cholesterol biomarker score of between about 26-50 and severe cardiovascular disease if the individual has an LDL cholesterol biomarker score of between about 0-25. The individual is determined to have mild diabetes risk if the individual has an serum A1C biomarker score of between about 51-75, moderate diabetes if the individual has a serum A1C biomarker score of between about 26-50 and severe diabetes if the individual has an serum A1C biomarker score of between about 0-25. The individual is determined to have mild excessive alcohol consumption if the individual has an alcohol biomarker score of between about 51-75, moderate excessive alcohol consumption if the individual has an alcohol biomarker score of between about 26-50 and severe excessive alcohol consumption if the individual has an alcohol biomarker score of between about 0-25. The individual is determined to have mild hypertension risk if the individual has a systolic blood pressure biomarker score of between about 51-75, moderate hypertension if the individual has a systolic blood pressure biomarker score of between about 26-50 and severe hypertension if the individual has a systolic blood pressure biomarker score of between about 0-25. The individual is determined to have mild poor nutrition risk if the individual has a glycemic food intake or nutrient dense food intake biomarker score of between about 51-75, moderate poor nutrition if the individual has a glycemic food intake or nutrient dense food intake biomarker score of between about 26-50 and severe poor nutrition if the individual has a glycemic food intake or nutrient dense food intake biomarker score of between about 0-25. The individual is determined to have mild obesity if the individual has a waist to height ratio biomarker score of between about 51-75, moderate obesity if the individual has a waist to height ratio biomarker score of between about 26-50 and severe obesity if the individual has a waist to height ratio biomarker score of between about 0-25. The individual is determined to have mild smoking if the individual has a smoking biomarker score of between about 51-75, moderate smoking risk if the individual has a smoking biomarker score of between about 26-50 and severe smoking risk if the individual has an smoking biomarker score of between about 0-25.

The recommendation is for one or more lifestyle and/or behavioral changes that the individual adopts to reduce the likelihood of developing one or more of cardiovascular disease, diabetes, excessive alcohol consumption, hypertension, poor nutrition, obesity, and/or smoking. In some embodiments, following the recommendation reduces the likelihood of an individual with a mild cardiovascular disease biomarker score of between about 51-75 for LDL cholesterol from progressing to a moderate cardiovascular disease biomarker score of between about 26-50 for LDL cholesterol and/or a severe cardiovascular disease biomarker score of between about 0-25 for LDL cholesterol. In other embodiments, following the recommendation reduces the likelihood of an individual with a mild diabetes biomarker score of between about 51-75 for serum A1C from progressing to a moderate diabetes biomarker score of between about 26-50 serum A1C and/or a severe diabetes biomarker score of between about 0-25 serum A1C. In other embodiments, following the recommendation reduces the likelihood of an individual with a mild excessive alcohol consumption biomarker score of between about 51-75 for alcohol from progressing to a moderate excessive alcohol consumption biomarker score of between about 26-50 for alcohol and/or a severe excessive alcohol consumption biomarker score of between about 0-25 for alcohol. In further embodiments, following the recommendation reduces the likelihood of an individual with a mild hypertension biomarker score of between about 51-75 for systolic blood pressure from progressing to a moderate hypertension biomarker score of between about 26-50 for systolic blood pressure and/or a severe hypertension biomarker score of between about 0-25 for systolic blood pressure. In other embodiments, following the recommendation reduces the likelihood of an individual with a mild poor nutrition risk biomarker score of between about 51-75 for glycemic food intake or nutrient dense food intake from progressing to a moderate poor nutrition risk biomarker score of between about 26-50 for glycemic food intake or nutrient dense food intake and/or a severe poor nutrition risk biomarker score of between about 0-25 for glycemic food intake or nutrient dense food intake. In some embodiments, following the recommendation reduces the likelihood of an individual with a mild obesity biomarker score of between about 51-75 for waist to height ratio from progressing to a moderate obesity biomarker score of between about 26-50 for waist to height ratio and/or a severe obesity biomarker score of between about 0-25 for waist to height ratio. In other embodiments, following the recommendation reduces the likelihood of an individual with a mild smoking biomarker score of between about 51-75 for smoking from progressing to a moderate smoking biomarker score of between about 26-50 for smoking and/or a severe smoking biomarker score of between about 0-25 for smoking.

Treating or Aiding in the Reduction of a Physiological Condition in an Individual that has a Physiological Condition

Further provided herein are methods for treating and/or aiding in the reduction of a physiological condition of an individual with one or more physiological conditions. The method involves receiving, by one or more processors, a score for each biomarker of one or more biomarkers for an individual and a severity for each biomarker, the severity being one of healthy, mild, moderate or severe; retaining, by the one or more processors, at least one biomarker within the one or more biomarkers that corresponds to a predetermined set of severities, the retaining encompassing removing, by the one or more processors, of at least one biomarker within the one or more biomarkers that neither has a severity assigned to it nor has a severity that does not fall within the predetermined set of severities; and determining, by the one or more processors, a physiological condition associated with the score of each retained biomarker based on the severity of the biomarker score; determining, by the one or more processors, a recommendation for improving the physiological condition; and sending, by the one or more processors, the recommendation to a computing application, the recommendation being used to treat the physiological condition associated with the score of each retained biomarker.

The physiological condition can be one or more of cardiovascular disease, diabetes, excessive alcohol consumption, hypertension, poor nutrition, obesity, and/or smoking. Each biomarker is assigned a severity score that ranges from 1-100. The predetermined set of severities for treating an individual with one or more physiological conditions (for example, cardiovascular disease, diabetes, excessive alcohol consumption, hypertension, poor nutrition, obesity, and/or smoking) are mild, moderate, and/or severe. For example, for an individual with a severity of mild, moderate, or severe for cardiovascular disease, following the recommendation will raise the individual's LDL cholesterol biomarker score by any of 1-10, 5-15, 10-20, 15-25, 20-30, 25-35, 30-40, 35-45, 40-50, 45-55, 50-60, 55-65, 60-70, 65-75, 70-80, 75-85, or 80-90 points, such as any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 or more points.

For an individual with a severity of mild, moderate, or severe for diabetes, following the recommendation will raise the individual's serum A1C biomarker score by any of 1-10, 5-15, 10-20, 15-25, 20-30, 25-35, 30-40, 35-45, 40-50, 45-55, 50-60, 55-65, 60-70, 65-75, 70-80, 75-85, or 80-90 points, such as any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 or more points.

For an individual with a severity of mild, moderate, or severe for excessive alcohol consumption, following the recommendation will raise the individual's alcohol biomarker score by any of about 1-10, 5-15, 10-20, 15-25, 20-30, 25-35, 30-40, 35-45, 40-50, 45-55, 50-60, 55-65, 60-70, 65-75, 70-80, 75-85, or 80-90 points, such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 or more points.

For an individual with a severity of mild, moderate, or severe for hypertension, following the recommendation will raise the individual's systolic blood pressure biomarker score by any of 1-10, 5-15, 10-20, 15-25, 20-30, 25-35, 30-40, 35-45, 40-50, 45-55, 50-60, 55-65, 60-70, 65-75, 70-80, 75-85, or 80-90 points, such as any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 or more points.

For an individual with a severity of mild, moderate, or severe for hypertension, following the recommendation will raise the individual's systolic blood pressure biomarker score by any of 1-10, 5-15, 10-20, 15-25, 20-30, 25-35, 30-40, 35-45, 40-50, 45-55, 50-60, 55-65, 60-70, 65-75, 70-80, 75-85, or 80-90 points, such as any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 or more points.

For an individual with a severity of mild, moderate, or severe for poor nutrition, following the recommendation will raise the individual's glycemic food intake or nutrient dense food intake biomarker score by any of 1-10, 5-15, 10-20, 15-25, 20-30, 25-35, 30-40, 35-45, 40-50, 45-55, 50-60, 55-65, 60-70, 65-75, 70-80, 75-85, or 80-90 points, such as any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 or more points.

For an individual with a severity of mild, moderate, or severe for obesity, following the recommendation will raise the individual's waist to height ratio biomarker score by any of 1-10, 5-15, 10-20, 15-25, 20-30, 25-35, 30-40, 35-45, 40-50, 45-55, 50-60, 55-65, 60-70, 65-75, 70-80, 75-85, or 80-90 points, such as any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 or more points.

Finally, For an individual with a severity of mild, moderate, or severe for smoking, following the recommendation will raise the individual's smoking biomarker score by any of 1-10, 5-15, 10-20, 15-25, 20-30, 25-35, 30-40, 35-45, 40-50, 45-55, 50-60, 55-65, 60-70, 65-75, 70-80, 75-85, or 80-90 points, such as any of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 or more points.

The current subject matter can be further understood by reference to the following examples, which are provided by way of illustration and are not in any way meant to be limiting.

### Exemplary Implementations

The following is a discussion of exemplary non-limiting implementations of the current matter system(s), device(s), and/or method(s). A user who wishes to improve or monitor one or more health-related conditions receives an email invitation to use the system (as shown in FIG. 18). The user clicks on the “Get Started” link and a graphical user interface showing an overview of the application is then displayed (as shown in FIGS. 19-20). The user is then invited to input data relevant to one or more biomarkers (age, gender, weight, height, systolic blood pressure, serum A1C, LDL cholesterol, anti-cholesterol medications; as shown in FIGS. 21-23) to receive a health score (as shown in FIG. 4). These biomarkers are used to create a health profile for the user.

FIG. 24 illustrates a screenshot showing a health score generated for each biomarker for a user as well as the overall health score (OHS). The score for each biomarker represents the severity of a particular disease or physiological condition known to be associated with a particular biomarker (for example, the serum A1C biomarker is associated with diabetes). A biomarker score of 76-100 indicates that the user is considered healthy for a physiological condition that correlate with that particular biomarker (for example, the nutrient dense food biomarker correlates with the physiological condition of poor nutrition). A biomarker score of 51-75 indicates that the user is considered at low risk for that particular biomarker. A biomarker score of 26-50 indicates that the user is considered at moderate risk for that particular biomarker. A biomarker score of 0-25 indicates that the user is considered at severe risk for that particular biomarker. Based on the user's biomarker scores, a recommendation for behavioral and/or lifestyle modification is provided to the user. The user periodically updates his or her health profile for data associated with each biomarker to track improvement or worsening of the biomarker score (as shown in FIGS. 25-26).

The application periodically provides the user challenges to help improve one or more biomarker score (as shown in FIGS. 27-30). These challenges are recommended for the user based on the user's biomarker scores and overall score. The application additionally contains a library containing articles, recipes, videos, pictures, and other data useful for improving biomarker scores which are made available to a user. FIG. 31 illustrates a graphical user interface of the application 123 displaying a portion of the library of articles, recipes, videos, pictures, and any other data that are stored in the system 102 and made available to a user. FIG. 32 illustrates a graphical user interface of the application 123 displaying another portion of the library of articles, recipes, videos, pictures, and any other data that are stored in the system 102 and made available to a user. FIG. 33 illustrates another graphical user interface of the application 123 displaying biomarker scores 1104 for the user and an overall health score 1106 for that user.

Note that the terms user and individual have been used interchangeably at several places herein. Various implementations of the subject matter described herein can be realized/implemented in digital electronic circuitry, integrated circuitry, specially designed application specific integrated circuits (ASICs), computer hardware, firmware, software, and/or combinations thereof. These various implementations can be implemented in one or more computer programs. These computer programs can be executable and/or interpreted on a programmable system. The programmable system can include at least one programmable processor, which can be a special purpose or a general purpose. The at least one programmable processor can be coupled to a storage system, at least one input device, and at least one output device. The at least one programmable processor can receive data and instructions from, and can transmit data and instructions to, the storage system, the at least one input device, and the at least one output device.

These computer programs (also known as programs, software, software applications or code) can include machine instructions for a programmable processor, and can be implemented in a high-level procedural and/or object-oriented programming language, and/or in assembly/machine language. As can be used herein, the term “machine-readable medium” can refer to any computer program product, apparatus and/or device (for example, magnetic discs, optical disks, memory, programmable logic devices (PLDs)) used to provide machine instructions and/or data to a programmable processor, including a machine-readable medium that can receive machine instructions as a machine-readable signal. The term “machine-readable signal” can refer to any signal used to provide machine instructions and/or data to a programmable processor.

To provide for interaction with a user, the subject matter described herein can be implemented on a computer that can display data to one or more users on a display device, such as a cathode ray tube (CRT) device, a liquid crystal display (LCD) monitor, a light emitting diode (LED) monitor, or any other display device. The computer can receive data from the one or more users via a keyboard, a mouse, a trackball, a joystick, or any other input device. To provide for interaction with the user, other devices can also be provided, such as devices operating based on user feedback, which can include sensory feedback, such as visual feedback, auditory feedback, tactile feedback, and any other feedback. The input from the user can be received in any form, such as acoustic input, speech input, tactile input, or any other input.

The subject matter described herein can be implemented in a computing system that can include at least one of a back-end component, a middleware component, a front-end component, and one or more combinations thereof. The back-end component can be a data server. The middleware component can be an application server. The front-end component can be a client computer having a graphical user interface or a web browser, through which a user can interact with an implementation of the subject matter described herein. The components of the system can be interconnected by any form or medium of digital data communication, such as a communication network. Examples of communication networks can include a local area network, a wide area network, internet, intranet, Bluetooth network, infrared network, or other networks.

The computing system can include clients and servers. A client and server can be generally remote from each other and can interact through a communication network. The relationship of client and server can arise by virtue of computer programs running on the respective computers and having a client-server relationship with each other.

Although a few variations have been described in detail above, other modifications can be possible. For example, the logic flows depicted in the accompanying figures and described herein do not require the particular order shown, or sequential order, to achieve desirable results. Other embodiments may be within the scope of the following claims.

What is claimed is:

1. A computer-implemented method comprising:
receiving, by one or more processors, one or more values corresponding to one or more biomarkers for a subject;
executing, by the one or more processors, a normalization routine to normalize each biomarker of the one or more biomarkers, the normalizing quantifying each biomarker on a corresponding preset scale to generate the normalized biomarker;
generating, by the one or more processors, a score for each normalized biomarker of the one or more biomarkers;
assigning, by the one or more processors, a predetermined weight for each normalized biomarker, the predetermined weight being obtained from a first database communicatively coupled to the one or more processors; and
computing, by the one or more processors, a health score for the subject based on the generated score for each normalized biomarker and the predetermined weight of each normalized biomarker.

2. The computer-implemented method of claim 1, wherein the one or more processors are located within a backend system.

3. The computer-implemented method of claim 1, wherein the one or more processors receive the one or more values of the one or more biomarkers for the subject from at least one of a computing application executed on a computing device operably coupled with the backend system via a communication network and a second database operably coupled to the one or more processors.

4. The computer-implemented method of claim 1, wherein a part of the one or more processors that receives the one or more values of the one or more biomarkers from the computing application is one of an application programming interface (API) module and a web module.

5. The computer-implemented method of claim 4, wherein a part of the one or more processors that performs the normalizing of each biomarker, the generating of the score for each biomarker, the obtaining of the predetermined weight for each biomarker, and the assigning of the predetermined weight for each biomarker is a scoring module operably coupled to the API module and the web module.

6. The computer-implemented method of claim 1, wherein at least one of the one or more biomarkers are input on a computing application executed on a computing device operably coupled with the one or more processors via a communication network.

7. The computer-implemented method of claim 6, wherein at least one of the one or more biomarkers are received from a second database storing a plurality of biomarkers previously input by the user on the computing application.

8. The computer-implemented method of claim 1, wherein the one or more biomarkers are selected from a group consisting of: cholesterol level, waist to height ratio, blood pressure, serum A1C levels, alcohol consumption, glycemic food intake, nutrient dense food intake, physical activity level, smoking, frequency and telomere length.

9. The computer-implemented method of claim 1, further comprising:
assigning, by the one or more processors, a severity to each biomarker of the one or more biomarkers, the severity being one of healthy, mild, moderate or severe; and
generating, by the one or more processors, a treatment recommendation based on the severity of each biomarker, on the score for each biomarker, and the score for the subject.

10. The computer-implemented method of claim 9, further comprising:
sending, by the one or more processors, the treatment recommendation to a computing device operably coupled with the one or more processors via a communication network.

11. The computer-implemented method of claim 10, wherein the treatment recommendation comprises at least one of: a text, an audio, and a video.

12. The computer-implemented method of claim 9, wherein the treatment recommendation is generated immediately after the receiving of the one or more values of the one or more biomarkers.

13. A method for reducing a likelihood of developing one or more physiological conditions, the method comprising:
receiving, by one or more processors, a score for each biomarker of one or more biomarkers for an individual and a severity for each biomarker, the severity being one of healthy, mild, moderate or severe;
removing, by the one or more processors, at least one biomarker within the one or more biomarkers that corresponds to a predetermined set of severities, the removed at least one biomarker within the one or more biomarkers having neither a severity assigned to it nor a severity that does not fall within the predetermined set of severities;
analyzing, by the one or more processors, the one or more biomarkers after the removing of the at least one biomarker to determine a physiological condition associated with the score of each biomarker of the one or more biomarkers after the removing of the at least one biomarker;
generating, by the one or more processors, a recommendation for improving the physiological condition; and
transmitting, by the one or more processors, the recommendation to a computing application, the recommendation being used to reduce the likelihood of the individual developing one or more physiological conditions.

14. The method of claim 13, wherein:
the physiological condition is cardiovascular disease and the biomarker is serum low density lipoprotein (LDL) level;
the physiological condition is diabetes and the biomarker is serum A1C level;
the physiological condition is hypertension and the biomarker is systolic blood pressure;
the physiological condition is obesity and the biomarker is waist to height ratio;
the physiological condition is poor activity and the biomarker is activity level;
the physiological condition is excessive alcohol and the biomarker is alcohol consumption;
the physiological condition is poor nutrition and the biomarker is high glycemic food intake and/or nutrient dense food intake; or
the physiological condition is smoking and the biomarker is smoking frequency.

15. The method of claim 13, wherein:
the physiological condition is cardiovascular disease and the recommendation for improving the physiological condition is selected from the group consisting of diet modification, increased activity level, decreased alcohol consumption, or smoking cessation;
the physiological condition is diabetes and the recommendation for improving the physiological condition is selected from the group consisting of diet modification, increased activity level, weight loss, or smoking cessation;
the physiological condition is hypertension and the recommendation for improving the physiological condition is selected from the group consisting of increased activity level, meditation, decreased alcohol consumption, or smoking cessation; or
the physiological condition is obesity and the recommendation for improving the physiological condition is selected from the group consisting diet modification, increased activity level, or weight loss.

16. The method of claim 14, wherein one or more of cardiovascular disease, diabetes, hypertension, or obesity have biomarker scores with severe or moderate severities.

17. The method of claim 16, further comprising determining, by the one or more processors, that the individual has a co-morbid physiological condition associated with one or more of:
cardiovascular disease, wherein the co-morbid physiological condition is one or more of poor activity, excessive alcohol, hypertension, obesity, or smoking when any of the co-morbid physiological conditions have biomarker scores with severe or moderate severities;
diabetes, wherein the co-morbid physiological condition is one or more of poor activity, cardiovascular disease, poor nutrition due to high glycemic food intake, hypertension, obesity, or smoking when any of the co-morbid physiological conditions have biomarker scores with severe or moderate severities;
hypertension, wherein the co-morbid physiological condition is one or more of poor activity, excessive alcohol, or smoking when any of the co-morbid physiological conditions have biomarker scores with severe or moderate severities; and
obesity wherein the co-morbid physiological condition is smoking when the co-morbid physiological condition has a biomarker score with a severe or moderate severity.

18. A non-transitory computer program product storing instructions that, when executed by at least one programmable processor, cause the at least one programmable processor to perform operations comprising:
receiving one or more values corresponding to one or more biomarkers for a subject;
executing a normalization routine to normalize each biomarker of the one or more biomarkers, the normalizing quantifying each biomarker on a corresponding preset scale to generate the normalized biomarker;
generating a score for each normalized biomarker of the one or more biomarkers;
assigning a predetermined weight for each normalized biomarker, the predetermined weight being obtained from a first database communicatively coupled to the at least one programmable processor; and
computing a health score for the subject based on the generated score for each normalized biomarker and the predetermined weight of each normalized biomarker.

19. The non-transitory computer program product of claim 18, wherein:
the one or more biomarkers comprise at least one of: cholesterol level, waist to height ratio, blood pressure, serum A1C levels, alcohol consumption, glycemic food intake, nutrient dense food intake, physical activity level, smoking frequency, and telomere length; and
the one or more biomarkers form a biomarker panel.

20. A method of aiding in the reduction of a physiological condition of an individual, the method comprising:
receiving, by one or more processors, one or more values of one or more biomarkers specific for hypertension for an individual;
normalizing, by the one or more processors, each biomarker of the one or more biomarkers;
generating, by the one or more processors, a score for each biomarker of the one or more biomarkers;
obtaining, by the one or more processors, a predetermined weight for each biomarker; and
computing, by the one or more processors, a health score for the individual based on the score for each biomarker and the predetermined weight for each biomarker.

21. A system comprising:
a frontend unit comprising one or more processors configured to receive values of a plurality of biomarkers from an application executed by a computing device of a user, generate a score for each biomarker, compute a severity associated with each biomarker, and generate an overall score for the user based on at least one of the score for each biomarker and the severity associated with each biomarker; and
a content unit operably coupled to the frontend unit, the content unit configured to store a library of data, the content unit comprising one or more processors configured to generate at least one treatment recommendation based on at least one of the score for each biomarker and the severity associated with each biomarker, the content unit configured to send the at least one treatment recommendation to the application.

22. The system of claim 21, wherein the application is configured to display the at least one treatment recommendation.

23. The system of claim 21, wherein:
the frontend unit comprises a first cluster of instances; and
the content unit comprises a second cluster of instances.

24. The system of claim 21, further comprising an integrations unit comprising one or more processors operably coupled to the frontend unit, the computing device of the user comprising a wearable device worn by the user, the integrations unit configured to receive at least one value of at least one biomarker of the plurality of biomarkers from the wearable device.

25. The system of claim 24, wherein the integrations unit comprises a cluster of instances.

26. The system of claim 21, further comprising an account and identity unit comprising one or more processors operably coupled to the frontend unit, the account and identity unit comprising authentication data associated with the user along with authentication data associated with a plurality of other users.

27. The system of claim 26, wherein the account and identity unit comprises a cluster of instances.

28. The system of claim 21, further comprising a secure health store unit comprising one or more processors operably coupled to the frontend unit, the secure health store unit storing the values of the one or more biomarkers for the user.

29. The system of claim 28, wherein the secure health store unit comprises a cluster of instances.

30. The system of claim 21, further comprising a notifications unit comprising one or more processors operably coupled to the frontend unit, the notifications unit configured to generate a notification configured to be sent to the computing device of the user.

31. The system of claim 30, wherein the notifications unit comprises a cluster of instances.

32. The system of claim 30, wherein the notification is sent via at least one of an email, a text message, and a social network message.

33. The system of claim 30, wherein the notification comprises an indication of the at least one treatment recommendation.

34. The system of claim 21, wherein the treatment recommendation comprises an automated scheduling of a visit to a clinician.
